(19) United States
(12) Patent Application Publication
(10) Pub. No.: US 2007/0049578 A1
(43) Pub. Date: Mar. 1, 2007

(54) Cinnamamide compounds as metabotropic glutamate receptor antagonists

(75) Inventors: Louise Edwards, Toronto (CA); Methvin Isaac, Toronto (CA); Abdelmalik Slassi, Toronto (CA); Tao Xin, Toronto (CA); Peter Dove, Toronto (CA)

Correspondence Address: BIRCH, STEWART, KOLASCH & BIRCH, LLP, P.O. BOX 747, 8110 GATEHOUSE ROAD, SUITE 500 EAST, FALLS CHURCH, VA, 22040-0747, US

(73) Assignees: AstraZeneca AB, Sodertalje (SE); NPS PHARMACEUTICALS, Salt Lake City, UT (US)

(21) Appl. No.: 11/503,182

(22) Filed: Aug. 14, 2006

Related U.S. Application Data
(60) Provisional application No. 60/710,608, filed on Aug. 24, 2005.

(51) Int. Cl. A61K 31/551; A61K 31/497; A61K 31/496; C07D 403/02

(52) U.S. Cl. 514/218; 544/360; 540/575; 544/357; 514/252.11; 514/253.12

(57) ABSTRACT

The invention relates to compounds of formula I or pharmaceutically acceptable salts or solvates thereof: 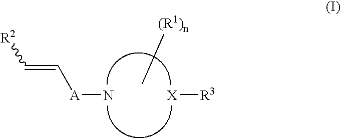
where R¹, R², R³, A, X, and n are defined in the description. The invention also includes pharmaceutical compositions and uses of, and processes of making the compounds, as well as methods of medical treatment of mGluR 5 mediated disorders.

### FIELD OF THE INVENTION

The present invention relates to a new class of compounds, to pharmaceutical formulations containing said compounds and to the use of said compounds in therapy. The present invention further relates to the process for the preparation of said compounds and to new intermediates prepared therein.

### BACKGROUND OF THE INVENTION

Glutamate is the major excitatory neurotransmitter in the mammalian central nervous system (CNS). Glutamate produces its effects on central neurons by binding to and thereby activating cell surface receptors. These receptors have been divided into two major classes, the ionotropic and metabotropic glutamate receptors, based on the structural features of the receptor proteins, the means by which the receptors transduce signals into the cell, and pharmacological profiles.

The metabotropic glutamate receptors (mGluRs) are G protein-coupled receptors that activate a variety of intracellular second messenger systems following the binding of glutamate. Activation of mGluRs in intact mammalian neurons elicits one or more of the following responses: activation of phospholipase C; increases in phosphoinositide (PI) hydrolysis; intracellular calcium release; activation of phospholipase D; activation or inhibition of adenyl cyclase; increases or decreases in the formation of cyclic adenosine monophosphate (cAMP); activation of guanylyl cyclase; increases in the formation of cyclic guanosine monophosphate (cGMP); activation of phospholipase A₂; increases in arachidonic acid release; and increases or decreases in the activity of voltage- and ligand-gated ion channels. Schoepp et al., Trends Pharmacol. Sci. 14:13 (1993), Schoepp, Neurochem. Int. 24:439 (1994), Pin et al., Neuropharmacology 34:1 (1995), Bordi and Ugolini, Prog. Neurobiol. 59:55 (1999). Molecular cloning has identified eight distinct mGluR subtypes, termed mGluR1 through mGluR8. Nakanishi, Neuron 13:1031 (1994), Pin et al., Neuropharmacology 34:1 (1995), Knopfel et al., J. Med. Chem. 38:1417 (1995). Further receptor diversity occurs via expression of alternatively spliced forms of certain mGluR subtypes. Pin et al., PNAS 89:10331 (1992), Minakami et al., BBRC 199:1136 (1994), Joly et al., J. Neurosci. 15:3970 (1995).

Metabotropic glutamate receptor subtypes may be subdivided into three groups, Group I, Group II, and Group III mGluRs, based on amino acid sequence homology, the second messenger systems utilized by the receptors, and by their pharmacological characteristics. Group I mGluR comprises mGluR1, mGluR5 and their alternatively spliced variants. The binding of agonists to these receptors results in the activation of phospholipase C and the subsequent mobilization of intracellular calcium.

Neurological, Psychiatric and Pain Disorders.

Attempts at elucidating the physiological roles of Group I mGluRs suggest that activation of these receptors elicits neuronal excitation. Various studies have demonstrated that Group I mGluRs agonists can produce postsynaptic excitation upon application to neurons in the hippocampus, cerebral cortex, cerebellum, and thalamus, as well as other CNS regions. Evidence indicates that this excitation is due to direct activation of postsynaptic mGluRs, but it also has been suggested that activation of presynaptic mGluRs occurs, resulting in increased neurotransmitter release. Baskys, Trends Pharmacol. Sci. 15:92 (1992), Schoepp, Neurochem. Int. 24:439 (1994), Pin et al., Neuropharmacology 34:1(1995), Watkins et al., Trends Pharmacol. Sci. 15:33 (1994).

Metabotropic glutamate receptors have been implicated in a number of normal processes in the mammalian CNS. Activation of mGluRs has been shown to be required for induction of hippocampal long-term potentiation and cerebellar long-term depression. Bashir et al., Nature 363:347 (1993), Bortolotto et al., Nature 368:740 (1994), Aiba et al., Cell 79:365 (1994), Aiba et al., Cell 79:377 (1994). A role for mGluR activation in nociception and analgesia also has been demonstrated, Meller et al., Neuroreport 4: 879 (1993), Bordi and Ugolini, Brain Res. 871:223 (1999). In addition, mGluR activation has been suggested to play a modulatory role in a variety of other normal processes including synaptic transmission, neuronal development, apoptotic neuronal death, synaptic plasticity, spatial learning, olfactory memory, central control of cardiac activity, waking, motor control and control of the vestibulo-ocular reflex. Nakanishi, Neuron 13: 1031 (1994), Pin et al., Neuropharmacology 34:1, Knopfel et al., J. Med. Chem. 38:1417 (1995).

Further, Group I metabotropic glutamate receptors and mGluR5 in particular, have been suggested to play roles in a variety of pathophysiological processes and disorders affecting the CNS. These include stroke, head trauma, anoxic and ischemic injuries, hypoglycemia, epilepsy, neurodegenerative disorders such as Alzheimer's disease and pain. Schoepp et al., Trends Pharmacol. Sci. 14:13 (1993), Cunningham et al., Life Sci. 54:135 (1994), Hollman et al., Ann. Rev. Neurosci. 17:31 (1994), Pin et al., Neuropharmacology 34:1 (1995), Knopfel et al., J. Med. Chem. 38:1417 (1995), Spooren et al., Trends Pharmacol. Sci. 22:331 (2001), Gasparini et al. Curr. Opin. Pharmacol. 2:43 (2002), Neugebauer Pain 98:1 (2002). Much of the pathology in these conditions is thought to be due to excessive glutamate-induced excitation of CNS neurons. Because Group I mGluRs appear to increase glutamate-mediated neuronal excitation via postsynaptic mechanisms and enhanced presynaptic glutamate release, their activation probably contributes to the pathology. Accordingly, selective antagonists of Group I mGluR receptors could be therapeutically beneficial, specifically as neuroprotective agents, analgesics or anticonvulsants.

Further, it has also been shown that mGluR5 antagonists are useful for the treatment of addictions or cravings (for drugs, tobacco, alcohol, any appetizing macronutrients or non-essential food items).

Recent advances in the elucidation of the neurophysiological roles of metabotropic glutamate receptors generally and Group I in particular, have established these receptors as promising drug targets in the therapy of acute and chronic neurological and psychiatric disorders and chronic and acute pain disorders.

Gastro Intestinal Disorders

The lower esophageal sphincter (LES) is prone to relaxing intermittently. As a consequence, fluid from the stomach can pass into the esophagus since the mechanical barrier is temporarily lost at such times, an event hereinafter referred to as “reflux”.

Gastro-esophageal reflux disease (GERD) is the most prevalent upper gastrointestinal tract disease. Current pharmacotherapy aims at reducing gastric acid secretion, or at neutralizing acid in the esophagus. The major mechanism behind reflux has been considered to depend on a hypotonic lower esophageal sphincter. However, e.g. Holloway & Dent (1990) Gastroenterol. Clin. N. Amer. 19, pp. 517-535, has shown that most reflux episodes occur during transient lower esophageal sphincter relaxations (TLESRs), i.e. relaxations not triggered by swallows. It has also been shown that gastric acid secretion usually is normal in patients with GERD.

The novel compounds according to the present invention are assumed to be useful for the inhibition of transient lower esophageal sphincter relaxations (TLESRs) and thus for treatment of gastro-esophageal reflux disorder (GERD).

The wording “TLESR”, transient lower esophageal sphincter relaxations, is herein defined in accordance with Mittal, R. K., Holloway, R. H., Penagini R., Blackshaw, L. A., Dent, J., 1995; Transient lower esophageal sphincter relaxation.

Gastroenterology 109, pp. 601-610.

The wording “reflux” is herein defined as fluid from the stomach being able to pass into the esophagus, since the mechanical barrier is temporarily lost at such times.

The wording “GERD”, gastro-esophageal reflux disease, is herein defined in accordance with van Heerwarden, M. A., Smout A. J. P. M., 2000; Diagnosis of reflux disease. Baillière's Clin. Gastroenterol. 14, pp. 759-774.

Appetite-Related Disorders

The prevalence of obesity is increasing. At present, more than half of the U.S. population is overweight. Obesity increases a person's risk of illness and death due to diabetes, stroke, coronary artery disease, high cholesterol, hypertension as well as kidney and gall bladder disorders. Furthermore, obesity may increase the risk for some types of cancer. It is also a risk factor for the development of osteoarthritis and sleep apnea.

It has been shown that mGluR5 antagonists are useful for the treatment or prevention of appetite-related disorders (e.g. binge eating, anorexia, bulimia and compulsive).

Because of their physiological and pathophysiological significance, there is a need for new potent mGluR agonists and antagonists that display a high selectivity for mGluR subtypes, particularly the Group I receptor subtype, most particularly the mGluR5 subtype.

The object of the present invention is to provide compounds exhibiting an activity at metabotropic glutamate receptors (mGluRs), especially at the mGluR5 receptor.

### SUMMARY OF THE INVENTION

One embodiment of the invention relates to compounds of formula I or a pharmaceutically acceptable salt or solvate thereof: 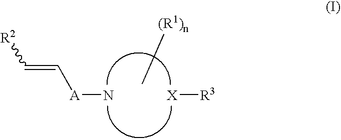
wherein 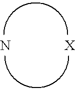
is a 5- to 7-membered ring that optionally contains, in addition to N and X, 1 to 2 heteroatoms that are independently selected from the group consisting of N, S, and O, and wherein 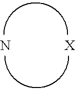
is optionally fused to a 5- to 7-membered ring that optionally contains one or more heteroatoms independently selected from the group consisting of N, O and S.
R¹, in each instance, is independently selected from the group consisting of F, Cl, Br, I, OH, CN, nitro, C₁₋₆-alkyl, OC₁₋₆-alkyl, C₁₋₆-alkylhalo, OC₁₋₆-alkylhalo, C₂₋₆-alkenyl, OC₂₋₆-alkenyl, C₂₋₆-alkynyl, OC₂₋₆-alkynyl, C₃₋₈-cycloalkyl, OC₃₋₈-cycloalkyl, C₁₋₆-alkylene-C₃₋₈-cycloalkyl, OC₁₋₆-alkylene-C₃₋₈-cycloalkyl, C₃₋₈-heterocycloalkyl, OC₃₋₈-heterocycloalkyl, C₁₋₆-alkylene-C₃₋₈-heterocycloalkyl, OC₁₋₆-alkylene-C₃₋₈-heterocycloalkyl, aryl, heteroaryl, C₁₋₆-alkylenearyl, C₁₋₆-alkyleneheteroaryl, OC₁₋₆-alkylenearyl, OC₁₋₆-alkyleneheteroaryl, C₁₋₆-alkyleneheterocycloalkyl, OC₁₋₆-alkyleneheterocycloalkyl, (CO)R⁴, O(CO)R⁴, O(CO)OR⁴, CO₂R⁴, O(CNR⁵)OR⁴, C₁₋₆-alkyleneOR⁴, OC₂₋₆-alkyleneOR⁴, C₁₋₆-alkylene(CO)R⁴, OC₁₋₆-alkylene(CO)R⁴, C₁₋₆-alkyleneCO₂R⁴, OC₁₋₆-alkyleneCO₂R⁴, C₁₋₆-alkylenecyano, OC₂₋₆-alkylenecyano, C₀₋₆-alkyleneNR⁴R⁵, OC₂₋₆-alkyleneNR⁴R⁵, C₁₋₆-alkylene(CO)NR⁴R⁵, OC₁₋₆-alkylene(CO)NR⁴R⁵, C₀₋₆-alkyleneNR⁴(CO)R⁵, OC₂₋₆-alkyleneNR⁴(CO)R⁵, Co-6-alkyleneNR⁴(CO)NR⁴R⁵, OC₁₋₆-alkyleneNR⁴(CO)NR⁴R⁵, C₀₋₆-alkyleneSR⁴, OC₂₋₆-alkyleneSR⁴, Co-6-alkylene(SO)R⁴, OC₂₋₆-alkylene(SO)R⁴, C₀₋₆-alkyleneSO₂R⁴, OC₂₋₆-alkyleneSO₂R⁴, C₀₋₆-alkylene(SO₂)NR⁴R⁵, OC₂₋₆-alkylene(SO₂)NR⁴R, C₀₋₆-alkyleneNR⁴(SO₂)R⁵, OC₂₋₆-alkyleneNR⁴(SO₂)R⁵, C₀₋₆-alkyleneNR⁴(SO₂)NR⁴R⁵, OC₂₋₆-alkyleneNR⁴(SO₂)NR⁴R⁵, (CO)NR⁴R⁵, O(CO)NR⁴R⁵, NR⁴OR⁵, C₁₋₆-alkyleneNR⁴OR⁵, OC₁₋₆-alkyleneNR⁴OR⁵, C₀₋₆-alkyleneNR⁴(CO)OR⁵, OC₂₋₆-alkyleneNR⁴(CO)OR⁵ and SO₃R⁴.
R² is a 5- to 7-membered ring that optionally contains one or more heteroatoms that are independently selected from the group consisting of N, O and S, wherein the 5- to 7-membered ring is optionally substituted with one or more substituents that are selected from the group consisting of F, Cl, Br, I, OH, nitro, C₁₋₆-alkyl, C₁₋₆-alkylhalo, OC₁₋₆-alkyl, OC₁₋₆-alkylhalo, —CN, aryl, heteroaryl, CO₂R⁴, NR⁴R⁵, SR⁴, S(O)R⁴ and SO₂R⁴.
R³ is selected from the group consisting of:
C(Y)YR⁴;
phenyl that is substituted at the 2-position, 4-position, or both, with one or more substituents selected from the group consisting of F, Cl, Br, I, nitro, C₁₋₆-alkyl, C₁₋₆-alkylhalo, OC₁₋₆-alkyl, OC₁₋₆-alkylhalo, and —CN; and
a 5- or 6-membered heterocyclic ring having a N atom in the ring at the position adjacent to the point of attachment and said ring is optionally substituted with one or more substituents selected from the group consisting of F, Cl, Br, I, OH, nitro, C₁₋₆-alkyl, C₁₋₆-alkylhalo, OC₁₋₆-alkyl, OC₁₋₆-alkylhalo, —N, aryl, heteroaryl, CO₂R⁴, SR⁴, S(O)R⁴ and SO₂R⁴, wherein when R³ is a 6-membered ring the substituents are located para to the point of attachment, or ortho to the point of attachment or both.
   R⁴ and R⁵ are independently selected from the group consisting of H, C₁₋₆-alkyl, C₁₋₆-alkylhalo, C₂₋₆-alkenyl, C₂₋₆-alkynyl, C₃₋₈-cycloalkyl, C₁₋₆-alkyl-C₃₋₈-cycloalkyl, aryl, C₁₋₆-alkylenearyl, C₁₋₆-alkylene-heterocycloalkyl, heteroaryl and C₁₋₆alkyleneheteroaryl, wherein any cyclic moiety is optionally fused to a 5- to 7-membered ring that may contain one or more heteroatoms independently selected from the group consisting of C, N, O and S.

A is selected from the group consisting of —CR⁴R⁵—, —C(O)—, —C(NR⁴)— and —C(S)—.

X is selected from the group consisting of C and N, wherein when X is carbon, then X, together with one adjacent carbon atom in the ring 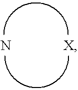
may form a double bond.

Y is selected from the group consisting of O, NR⁴ and S.

Variable n is an integer that is selected from the group consisting of 0, 1, 2, 3, 4, and 5.

The compound of formula I is subject to the proviso that the compound is not:
1-[4-(3-Nitro-pyridin-2-yl)-piperazin-1-yl]-3-phenyl-propenone.
1-[4-(3-methyl-2-pyridinyl)-piperazin-1-yl]-3-(4-trifluoromethylphenyl)-prop-2-en-1-one, or
1-[4-(3-trifluoromethyl-2-pyridinyl)-piperazin-1-yl]-3-(4-isopropylphenyl)-prop-2-en-1-one.

Another embodiment is a pharmaceutical composition comprising as active ingredient a therapeutically effective amount of the compound according to formula I, in association with one or more pharmaceutically acceptable diluents, excipients and/or inert carriers.

Other embodiments, as described in more detail below, relate to a compound according to formula I for use in therapy, in treatment of mGluR 5 mediated disorders, in the manufacture of a medicament for the treatment of mGluR5 mediated disorders.

Still other embodiments relate to a method of treatment of mGluR5 mediated disorders, comprising administering to a mammal a therapeutically effective amount of the compound according to formula I.

In another embodiment, there is provided a method for inhibiting activation of mGluR5 receptors, comprising treating a cell containing said receptor with an effective amount of the compound according to formula I.

### DETAILED DESCRIPTION OF THE PREFERRED EMBODIMENTS

The present invention is based upon the discovery of compounds that exhibit activity as pharmaceuticals, in particular as antagonists of metabotropic glutamate receptors. More particularly, the compounds of the present invention exhibit activity as antagonists of the mGluR5 receptor and, therefore, are useful in therapy, in particular for the treatment of neurological, psychiatric, pain, and gastrointestinal disorders,

Definitions

Unless specified otherwise within this specification, the nomenclature used in this specification generally follows the examples and rules stated in Nomenclature of Organic Chemistry, Sections A, B, C, D, E, F, and H, Pergamon Press, Oxford, 1979, which is incorporated by references herein for its exemplary chemical structure names and rules on naming chemical structures. Optionally, a name of a compound may be generated using a chemical naming program: ACD/ChemSketch, Version 5.09/September 2001, Advanced Chemistry Development, Inc., Toronto, Canada.

The term “alkyl” as used herein means a straight- or branched-chain hydrocarbon radical having from one to six carbon atoms, and includes methyl, ethyl, propyl, isopropyl, t-butyl and the like.

The term “alkenyl” as used herein means a straight- or branched-chain alkenyl radical having from two to six carbon atoms, and includes ethenyl, 1-propenyl, 1-butenyl and the like.

The term “alkynyl” as used herein means a straight- or branched-chain alkynyl radical having from two to six carbon atoms, and includes 1-propynyl (propargyl), 1-butynyl and the like.

The term “cycloalkyl” as used herein means a cyclic group (which may be unsaturated) having from three to seven carbon atoms, and includes cyclopropyl, cyclohexyl, cyclohexenyl and the like.

The term “heterocycloalkyl” as used herein means a three- to seven-membered cyclic group (which may be unsaturated) having at least one heteroatom selected from the group consisting of N, S and O, and includes piperidinyl, piperazinyl, pyrrolidinyl, tetrahydrofuranyl and the like.

The term “alkoxy” as used herein means a straight- or branched-chain alkoxy radical having from one to six carbon atoms and includes methoxy, ethoxy, propyloxy, isopropyloxy, t-butoxy and the like.

The term “halo” as used herein means halogen and includes fluoro, chloro, bromo, iodo and the like, in both radioactive and non-radioactive forms.

The term “alkylene” as used herein means a difunctional branched or unbranched saturated hydrocarbon radical having one to six carbon atoms, and includes methylene, ethylene, n-propylene, n-butylene and the like.

The term “alkenylene” as used herein means a difunctional branched or unbranched hydrocarbon radical having two to six carbon atoms and having at least one double bond, and includes ethenylene, n-propenylene, n-butenylene and the like.

The term “alkynylene” as used herein means a difunctional branched or unbranched hydrocarbon radical having two to six carbon atoms and having at least one triple bond, and includes ethynylene, n-propynylene, n-butynylene and the like.

The term “aryl” as used herein means an aromatic group having five to twelve atoms, and includes phenyl, naphthyl and the like.

The term “heteroaryl” means an aromatic group which includes at least one heteroatom selected from the group consisting of N, S and O, and includes groups and includes pyridyl, indolyl, furyl, benzofuryl, thienyl, benzothienyl, quinolyl, oxazolyl and the like.

The term “C₂₋₇alkanoyloxy” as used herein means a straight- or branched-chain alkanoyloxy radical (—OC(O)C₁₋₆-alkyl) having from two to seven carbon atoms, and includes acetoxy, propionyloxy, butyryloxy and the like.

The term “C₄₋₇cycloalkenyl” as used herein means an unsaturated carbocyclic group having from four to seven carbon atoms, and includes cyclopent-1-enyl, cyclohexenyl and the like.

The term “cycloalkyloxy” as used herein means a saturated carbocyclo-oxy radical having from three to seven carbon atoms, and includes cyclopropyloxy, cyclohexyloxy and the like.

The term “cycloalkylthio” as used herein means a saturated carbocycloalkylthio radical having from three to seven carbon atoms, and includes cyclopropylthio, cyclohexylthio and the like.

The term “alkanoyl” as used herein means a straight- or branched-chain alkanoyl radical having from two to seven atoms, and includes acetyl, propionyl, butyryl and the like.

The term “cycloalkenyl” as used herein means an unsaturated cylcloaklyl group having from four to seven carbon atoms, and includes cyclopent-1-enyl, cyclohex-1-enyl and the like.

The term “optionally substituted phenyl” as used herein means an unsubstituted phenyl radical or a phenyl radical substituted with one to three substituents independently selected from the group consisting of halo, OH, alkyl, alkoxy, alkylthio, CF₃ and CF₃O.

The term “optionally substituted phenoxy” as used herein means an unsubstituted phenoxy radical or a phenoxy radical substituted with one to three substituents independently selected from the group consisting of halo, OH, alkyl, alkoxy, alkylthio, CF₃ and CF₃O.

The term “optionally substituted thienyl” as used herein means an unsubstituted thienyl radical or a thienyl radical substituted with one or two substituents independently selected from the group consisting of halo, OH, alkyl, alkoxy, alkylthio, CF₃ and CF₃O.

The term “optionally substituted furanyl” as used herein means an unsubstituted furanyl radical or a furanyl radical substituted with one or two substituents independently selected from the group consisting of halo, OH, alkyl, alkoxy, alkylthio, CF₃ and CF₃O.

The term “optionally substituted benzyl” as used herein means an unsubstituted benzyl radical or a benzyl radical substituted on the phenyl ring with one to three substituents independently selected from the group consisting of halo, OH, alkyl, alkoxy, alkylthio, CF₃ and CF₃O.

The term “optionally substituted benzyloxy” as used herein means an unsubstituted benzyloxy radical or a benzyloxy radical substituted on the phenyl ring with one to three substituents independently selected from the group consisting of halo, OH, alkyl, alkoxy, alkylthio, CF₃ and CF₃O.

The terms “alkylaryl”, “alkylheteroaryl” and “alkylcycloalkyl” refer to an alkyl radical substituted with an aryl, heteroaryl or cycloalkyl group, and includes 2-phenethyl, 3-cyclohexyl propyl and the like.

The term “5- or 6-membered ring containing one or more atoms independently selected from C, N, O or S” includes aromatic and heteroaromatic rings, as well as carbocyclic and heterocyclic rings which may be saturated or unsaturated, and includes furyl, isoxazolyl, oxazolyl, pyrazolyl, pyridazinyl, pyridyl, pyrimidyl, pyrrolyl, thiazolyl, thienyl, imidazolyl, triazolyl, morpholinyl, piperazinyl, piperidyl, tetrahydropyranyl, phenyl, cyclohexyl, cyclopentyl, cyclohexanyl and the like.

The term “3- to 8-membered ring containing one or more atoms independently selected from C, N, O or S”, includes aromatic and heteroaromatic rings as well as carbocyclic and heterocyclic rings which may be saturated or unsaturated, and includes imidazolinyl, morpholinyl, piperazinyl, piperidyl, pyrazolinyl, pyrrolidinyl, pyrrolinyl, tetrahydropyranyl or thiomorpholinyl, tetrahydrothiopyranyl, furyl, pyrrolyl, isoxazolyl, isothiazolyl, oxazolyl, oxazolidinonyl, pyrazinyl, pyrazolyl, pyridazinyl, pyridyl, pyrimidyl, pyrrolyl, thiazolyl, thienyl, imidazolyl, triazolyl, phenyl, cyclopropyl, aziridinyl, cyclobutyl, azetidinyl, cyclopentyl, cyclopentenyl, cyclohexyl, cyclohexenyl, cycloheptyl, cycloheptenyl, cyclooctyl and cyclooctenyl.

The term “3- to 8-membered ring containing one or more atoms independently selected from C, N, O or S, which group may optionally be fused with a 5- or 6-membered ring containing one or more atoms independently selected from C, N, O or S”, includes aromatic and heteroaromatic rings as well as carbocyclic and heterocyclic rings which may be saturated or unsaturated, and includes naphthyl, chromyl, isochromyl, indanyl, benzoimidazolyl, tetralinyl, benzothiazolyl, benzofuryl, benzothienyl, indolyl, azaindolyl, benzimidazolyl, oxadiazolyl, thiadiazolyl, quinolinyl, quinoxalinyl and benzotriazolyl.

The term “pharmaceutically acceptable salt” means either an acid addition salt or a basic addition salt which is compatible with the treatment of patients.

A “pharmaceutically acceptable acid addition salt” is any non-toxic organic or inorganic acid addition salt of the base compounds represented by Formula I or any of its intermediates. Illustrative inorganic acids which form suitable salts include hydrochloric, hydrobromic, sulfuric and phosphoric acid and acid metal salts such as sodium monohydrogen orthophosphate and potassium hydrogen sulfate. Illustrative organic acids which form suitable salts include the mono-, di- and tricarboxylic acids. Illustrative of such acids are, for example, acetic, glycolic, lactic, pyruvic, malonic, succinic, glutaric, fumaric, malic, tartaric, citric, ascorbic, maleic, hydroxymaleic, benzoic, hydroxybenzoic, phenylacetic, cinnamic, salicylic, 2-phenoxybenzoic, p-toluenesulfonic acid and other sulfonic acids such as methanesulfonic acid and 2-hydroxyethanesulfonic acid. Either the mono- or di-acid salts can be formed, and such salts can exist in either a hydrated, solvated or substantially anhydrous form. In general, the acid addition salts of these compounds are more soluble in water and various hydrophilic organic solvents, and generally demonstrate higher melting points in comparison to their free base forms. The selection criteria for the appropriate salt will be known to one skilled in the art. Other non-pharmaceutically acceptable salts e.g. oxalates may be used for example in the isolation of compounds of Formula I for laboratory use, or for subsequent conversion to a pharmaceutically acceptable acid addition salt.

A “pharmaceutically acceptable basic addition salt” is any non-toxic organic or inorganic base addition salt of the acid compounds represented by Formula I or any of its intermediates. Illustrative inorganic bases which form suitable salts include lithium, sodium, potassium, calcium, magnesium or barium hydroxides. Illustrative organic bases which form suitable salts include aliphatic, alicyclic or aromatic organic amines such as methylamine, trimethyl amine and picoline or ammonia. The selection of the appropriate salt may be important so that an ester functionality, if any, elsewhere in the molecule is not hydrolyzed. The selection criteria for the appropriate salt will be known to one skilled in the art.

“Solvate” means a compound of Formula I or the pharmaceutically acceptable salt of a compound of Formula I wherein molecules of a suitable solvent are incorporated in a crystal lattice. A suitable solvent is physiologically tolerable at the dosage administered as the solvate. Examples of suitable solvents are ethanol, water and the like. When water is the solvent, the molecule is referred to as a hydrate.

The term “stereoisomers” is a general term for all isomers of the individual molecules that differ only in the orientation of their atoms in space. It includes mirror image isomers (enantiomers), geometric (cis/trans) isomers and isomers of compounds with more than one chiral centre that are not mirror images of one another (diastereomers).

The term “treat” or “treating” means to alleviate symptoms, eliminate the causation of the symptoms either on a temporary or permanent basis, or to prevent or slow the appearance of symptoms of the named disorder or condition.

The term “therapeutically effective amount” means an amount of the compound which is effective in treating the named disorder or condition.

Compounds

Compounds of the invention conform generally to formula I: 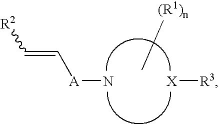
wherein R¹, R², R³, A, X, and n are defined hereinabove.

In one embodiment, X can be N. Thus, for example, 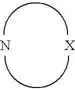
can be piperazinyl.

In another embodiment, R² can be selected from the group consisting of optionally substituted phenyl, pyridyl, and thienyl. Illustrative of R² is a phenyl group that is substituted with one or more substituents that are selected from the group consisting of F, Cl, Br, nitro, C₁₋₆-alkyl, C₁₋₆-alkylhalo, OC₁₋₆-alkyl, OC₁₋₆-alkylhalo, and —CN.

In other embodiments, R² is an optionally substituted pyridyl, such as, for example, optionally substituted 2-pyridyl.

In another embodiment, R² is optionally substituted thienyl. Illustrative of R² in this regard is an optionally substituted 3-thienyl group.

In still another embodiment, R¹ can be selected from the group consisting of C₁₋₆-alkyl, C₁₋₆-haloalkyl, —CN, —CO₂R₄, —CONR₄R₅, —C₁₋₆-alflyleneOR₄, —(CH₂)nNR₄, —C₁₋₆-ayleneCO₂R₄, and the following rings:

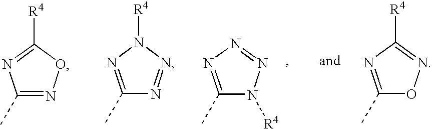

Another embodiment provides for R⁴ and R⁵ being independently selected from H and C₁₋₆-alkyl.

A further embodiment provides for R¹ being selected from the group consisting of C₁₋₆-alkyl, CN and CO₂R⁴, where R⁴ is C₁₋₆alkyl.

In one embodiment, R³ can be C(Y)YR⁴. In another embodiment R³ can be phenyl that is substituted at the 2-position, 4-position, or both, with one or more substituents selected from the group consisting of F, Cl, Br, I, nitro, C₁₋₆-alkyl, C₁₋₆-alkylhalo, OC₁₋₆-alkyl, OC₁₋₆-alkylhalo, and —N. Thus, for example, R³ can be phenyl that is substituted at the 2-position.

In another embodiment, R³ is a 5- or 6-membered heterocyclic ring having an N atom in the ring adjacent to the point of attachment wherein the ring is optionally substituted with one or more substituents selected from the group consisting of F, Cl, Br, I, OH, nitro, C₁₋₆-alkyl, C₁₋₆-alkylhalo, OC₁₋₆-alkyl, OC₁₋₆-alkylhalo, —CN, aryl, heteroaryl, CO₂R⁴, SR⁴, S(O)R⁴ and SO₂R⁴. In this embodiment, when R³ is a 6-membered ring, the substituents are located para to the point of attachment or ortho to the point of attachment or both. Illustrative substituents include but are not limited to F, Cl, Br, I, nitro, C₁₋₆-alkyl, C₁₋₆-alkylhalo, OC₁₋₆-alkyl, OC₁₋₆-alkylhalo, and —CN. Specific examples of R³ include but are not limited to optionally substituted 2-pyrazinyl and optionally substituted 2-pyridyl.

In another embodiment, 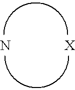
can be piperazinyl; R¹ can be selected from the group consisting of C₁₋₆-alkyl, C₁₋₆-haloalkyl, heteroaryl, —CN, —CO₂R₄, —CONR₄R₅, —C₁₋₆-alkyl-OR₄, —(CH₂)ₙNR₄, —C₁₋₆-alkyl-CO₂R₄; R² can be selected from the group consisting of optionally substituted phenyl, pyridyl, and thienyl; R³ can be a 2-pyridyl or 2-pyrazinyl ring that is optionally substituted in the position para to the point of attachment or ortho to the point of attachment or both, with one or more substituents selected from the group consisting of F, Cl, Br, I, nitro, C₁₋₆-alkyl, C₁₋₆-alkylhalo, OC₁₋₆-alkyl, OC₁₋₆-alkylhalo, and —CN; A can be —C(O)—; and the double bond to which R² is bound can be in the E configuration.

It will be understood by those of skill in the art that when compounds of the present invention contain one or more chiral centers, the compounds of the invention may exist in, and be isolated as, enantiomeric or diastereomeric forms, or as a racemic mixture. The present invention includes any possible enantiomers, diastereomers, racemates or mixtures thereof, of a compound of formula I. The optically active forms of the compound of the invention may be prepared, for example, by chiral chromatographic separation of a racemate or chemical or enzymatic resolution methodology, by synthesis from optically active starting materials or by asymmetric synthesis based on the procedures described thereafter.

It will also be appreciated by those of skill in the art that certain compounds of the present invention may exist as geometrical isomers, for example E and Z isomers of alkenes. The present invention includes any geometrical isomer of a compound of formula I. It will further be understood that the present invention encompasses tautomers of the compounds of formula I.

It will also be understood by those of skill in the art that certain compounds of the present invention may exist in solvated, for example hydrated, as well as unsolvated forms. It will further be understood that the present invention encompasses all such solvated forms of the compounds of formula I.

Within the scope of the invention are also salts of the compounds of formula I. Generally, pharmaceutically acceptable salts of compounds of the present invention are obtained using standard procedures well known in the art, for example, by reacting a sufficiently basic compound, for example an alkyl amine with a suitable acid, for example, HCl or acetic acid, to afford a salt with a physiologically acceptable anion. It is also possible to make a corresponding alkali metal (such as sodium, potassium, or lithium) or an alkaline earth metal (such as a calcium) salt by treating a compound of the present invention having a suitably acidic proton, such as a carboxylic acid or a phenol, with one equivalent of an alkali metal or alkaline earth metal hydroxide or alkoxide (such as the ethoxide or methoxide), or a suitably basic organic amine (such as choline or meglumine) in an aqueous medium, followed by conventional purification techniques. Additionally, quaternary ammonium salts can be prepared by the addition of alkylating agents, for example, to neutral amines.

In one embodiment of the present invention, the compound of formula I may be converted to a pharmaceutically acceptable salt or solvate thereof, particularly, an acid addition salt such as a hydrochloride, hydrobromide, phosphate, acetate, fumarate, maleate, tartrate, citrate, methanesulphonate or p-toluenesulphonate.

Specific examples of the present invention include the following compounds, their pharmaceutically acceptable salts, hydrates, solvates, optical isomers, and combinations thereof:

| Example | Compound | Name |
|---|---|---|
| 11.1 | 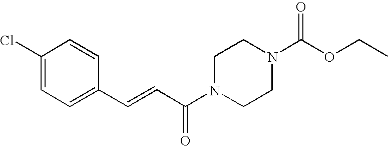 | ethyl 4-[(2E)-3-(4-chloro- phenyl prop-2-enoyl]piperazine-1-carboxylate |
| 11.2 | 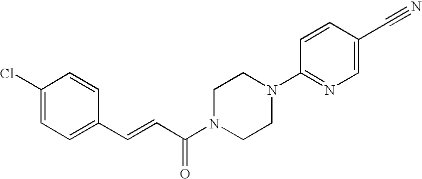 | 6-{4-[(2E)-3-(4- chiorophenyl) prop-2- enoyl]piperazin-1-yl}nicotinonitrile |
| 11.3 | 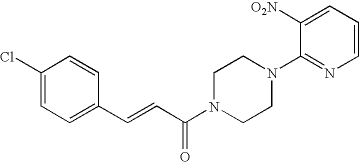 | 1-[(2E)-3-(4-chlorophenyl) prop-2-enoyl]-4-(3-mtro- pyridin-2-yl)piperazine |
| 11.4 | 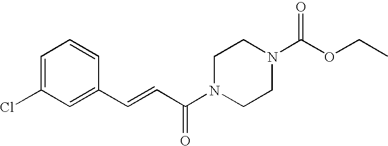 | ethyl 4-[(2E)-3-(3-chloro- phenyl)prop-2-enoyl]piperazine-1-carboxylate |
| 11.5 | 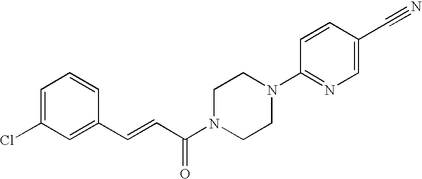 | 6-{4-[(2E)-3-(3-chloro- phenyl)prop-2-enoyl]piperazin-1-yl}nicotinonitrile |
| 11.6 | 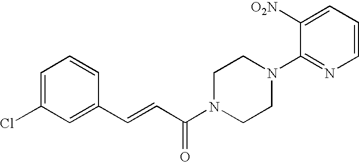 | 1-[(2E)-3-(3-chlorophenyl) prop-2-enoyl]-4-(3-nitro- pyridin-2-yl)piperazine |
| 11.7 | 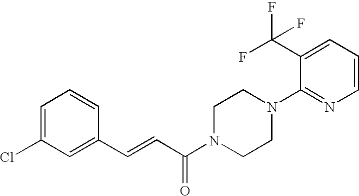 | 1-[(2E)-3-(3-chlorophenyl) prop-2-enoyl]-4-[3- (trifluoro-methyl)pyridin-2- yl]piperazine |
| 11.8 | 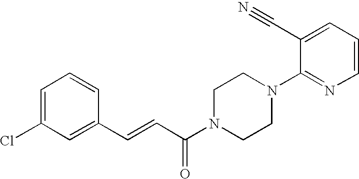 | 2-{4-[(2E)-3-(3- chiorophenyl) prop-2- enoyl]piperazin-1-yl}nicotinonhtrile |
| 11.9 | 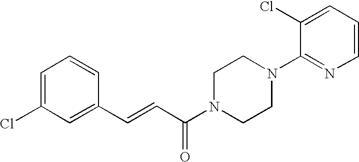 | 1-[(2E)-3-(3-chlorophenyl) prop-2-enoyl]-4-(3-chloro- pyridin-2-yl)piperazine |
| 11.10 | 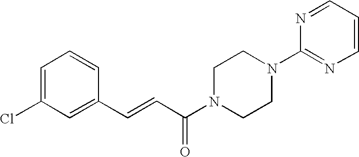 | 2-}4-[(2E)-3-(3- chlorophenyl) prop-2- enoyl]piperazin-1-yl}pyrimidine |
| 11.11 | 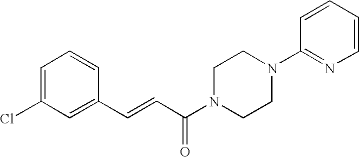 | 1-[(2E)-3-(3-chlorophenyl) prop-2-enoyl]-4-pyridin-2-yl piperazine |
| 11.12 | 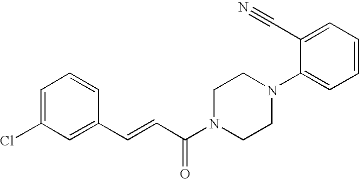 | 2-{4-[(2E)-3-(3- chlorophenyl) prop-2- enoyl]piperazin-1-yl}benzonitrile |
| 11.13 | 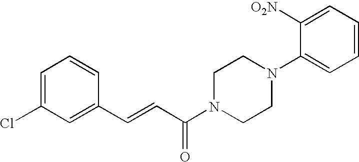 | 1-[(2E)-3-(3-chlorophenyl) prop-2-enoyl]-4-(2-nitro- phenyl)piperazine |
| 11.14 | 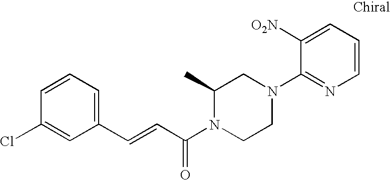 | (2S)-1-[(2E)-3-(3-chloro- phenyl)prop-2-enoyl]-2- methyl-4-(3-nitropyridin-2- yl) piperazine |
| 11.15 | 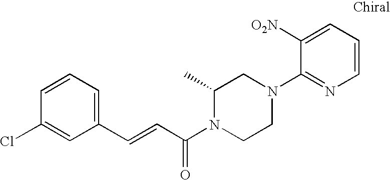 | (2R)-1-[(2E)-3-(3-chloro- phenyl)prop-2-enoyl]-2- methyl-4-(3-nitropyridin-2- yl) piperazine |
| 11.16 | 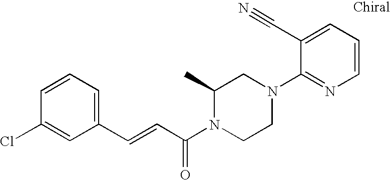 | 2-{(3S)-4-[(2E)-3-(3- chlorophenyl)prop-2-enoyl]- 3-methylpiperazin-1-yl}nicotinonitrile |
| 11.17 | 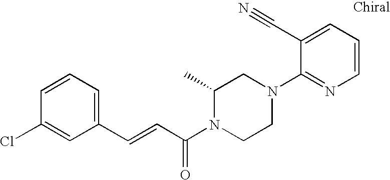 | 2-{(3R)-4-[(2E)-3-(3-chloro- phenyl)prop-2-enoyl]-3- methylpiperazrn-1-yl}nicotinonitrile |
| 11.18 | 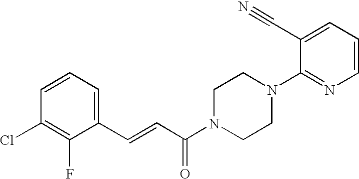 | 2-{4-[(2E)-3-(3-chloro-2- fluorophenyl)prop-2-enoyl]piperazin-1-yl}nicotinonitrile |
| 11.19 | 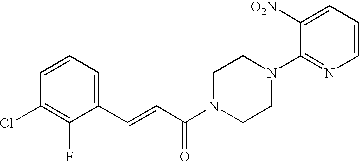 | 1-[(2E)-3-(3-chloro-2-fluoro- phenyl)prop-2-enoyl]-4-(3- nitropyridin-2-yI)piperazine |
| 11.20 | 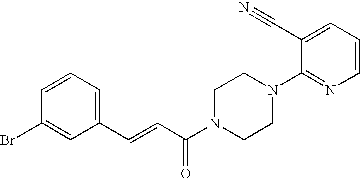 | 2-{4-[(2E)-3-(3-bromo- phenyl)prop-2-enoyl]piperazin-1-yl}nicotinomtrile |
| 11.21 | 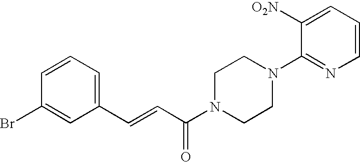 | 1-[(2E)-3-(3-bromo- phenyl)prop-2-enoyl]-4-(3- nitropyridin-2-yl)piperazine |
| 11.22 | 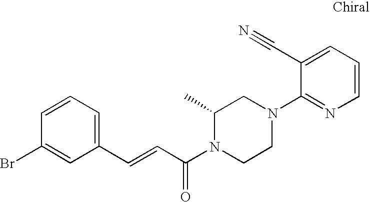 | 2-{(3R)-4-[(2E)-3-(3-bromo- phenyl)prop-2-enoyl]-3- methylpiperazin-1-yl}nicotinonitrile |
| 11.23 | 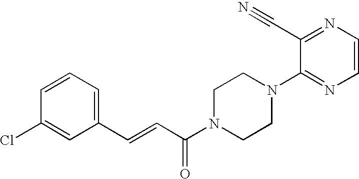 | 3-{4-[(2E)-3-(3-chloro- phenyl)prop-2-enoyl]piperazin-1-yl}pyrazine-2- carbonitrile |
| 11.24 | 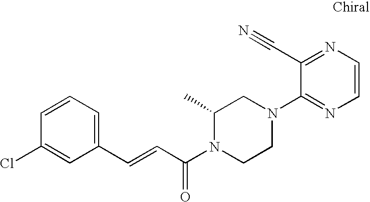 | 3-{(3R)-4-[(2E)-3-(3-chloro- phenyl)prop-2-enoyl]-3- methylpiperazin-1-yl}pyrazine-2-carbonitrile |
| 11.25 | 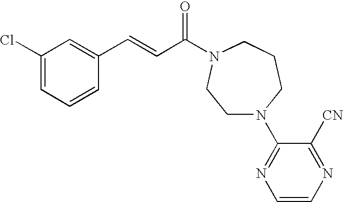 | 3-{4-[(2E)-3-(3-chloro- phenyl)prop-2-enoyl]-1,4- diazepan-1-yl}pyrazine-2- carbonitrile |
| 11.26 | 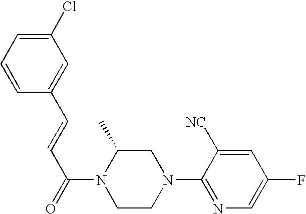 | 2-{(3R)-4-[(2E)-3-(3-chloro- phenyl)prop-2-enoyl]-3- methylpiperazin-1-yl}-5- fluoronicotinonitrile |
| 11.27 | 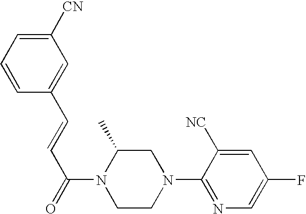 | 2-{(3R)-4-[(2E)-3-(3-cyano- phenyl)prop-2-enoyl]-3- methylpiperazin-1-yl}-5- fluoronicotinonitrile |
| 11.28 | 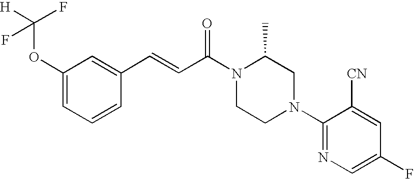 | 2-((3R)-4-{(2E)-3-[3- (difluoromethoxy)phenyl]prop- 2-enoyl}-3- methylpiperazin-1-yl)-5- fluoronicotinonitrile |
| 11.29 | 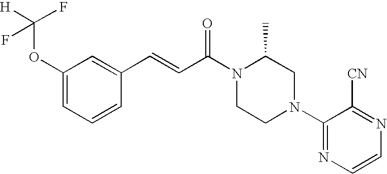 | 3-((3R)-4-{(2E)-3-[3- (difluoromethoxy)phenyl]prop- 2-enoyl}-3- methylpiperazin-1- yl)pyrazine-2-carbonitrile |
| 11.30 | 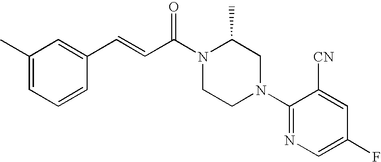 | 5-fluoro-2-{(3R)-3-methyl- 4-[(2E)-3-(3- methylphenyl)prop-2- enoyl]piperazin-1-yl}nicotinonitrile |
| 11.31 | 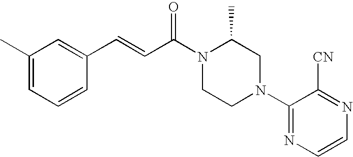 | 3-{(3R)-3-methyl-4-[(2E)-3- (3-methylphenyl)prop-2- enoyl]piperazin-1- yl}pyrazine-2-carbonitrile |
| 11.32 | 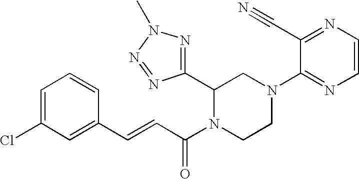 | 3-[4-[(2E)-3-(3- chlorophenyl) prop-2-enoyl]- 3-(2-methyl-2H-tetrazol-5- yl)piperazin-1-yl]pyrazine |
| 11.33 | 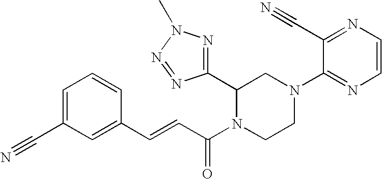 | 3-[4-[(2E)-3-(3- cyanophenyl) prop-2-enoyl]- 3-(2-methyl-2H-tetrazol-5- yl)piperazin-1-yl]pyrazine- 2-carbonitrile |
| 11.34 | 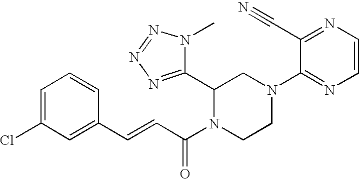 | 3-[4-[(2E)-3-(3- chlorophenyl) prop-2-enoyl]- 3-(1-methyl-1H-tetrazol-5- yl)piperazin-1-yl]pyrazine- 2-carbonitrile |
| 11.35 | 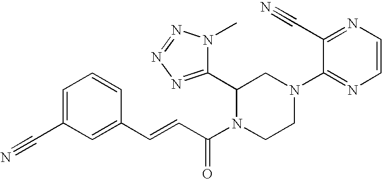 | 3-[4-[(2E)-3-(3- cyanophenyl) prop-2-enoyl]- 3-(1-methyl-1H-tetrazol-5- yl)piperazin-1-yl]pyrazine- 2-carbonitrile |
| 13.1 | 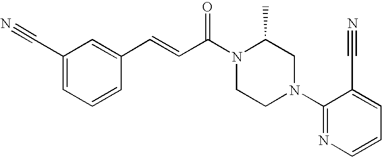 | 2-{(3R)-4-[(2E)-3-(3- cyanophenyl)prop-2-enoyl]- 3-methylpiperazin-1- yl}pyridine-3-carbonitrile |
| 13.2 | 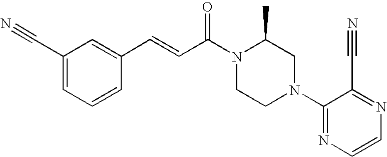 | 3-{(3S)-4-[(2E)-3-(3- cyanophenyl)prop-2-enoyl]- 3-methylpiperazin-1- yl}pyrazine-2-carbonitrile |
| 13.3 | 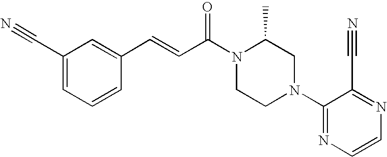 | 3-{(3R)-4-[(2E)-3-(3- cyanophenyl)prop-2-enoyl]- 3-methylpiperazin-1- yl}pyrazine-2-carbonitrile |
| 14.1 | 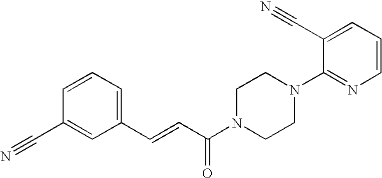 | 2-{4-[(2E)-3-(3- cyanophenyl)prop-2- enoyl]piperazine-1- yl}nicotinonitrile |
| 14.2 | 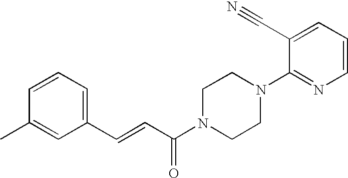 | 2-{4-[(2E)-3-(3- methylphenyl)prop-2- enoyl]piperazin-1- yl}nicotinonitrile |
| 14.3 | 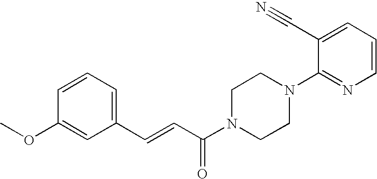 | 2-{4-[(2E)-3-(3- methoxyphenyl)prop-2- enoyl]piperazin-1- yl}nicotinonitrile |
| 14.4 | 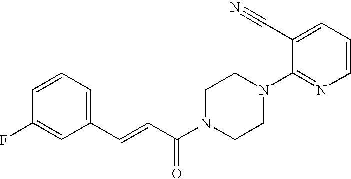 | 2-{4-[(2E)-3-(3- fluorophenyl)prop-2- enoyl]piperazin-1- yl}nicotinonitrile |
| 14.5 | 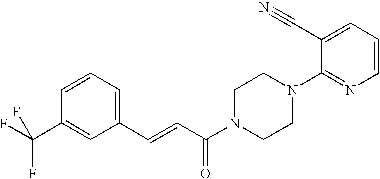 | 2-(4-{(2E)-3-[3- (trifluoromethyl) phenyl]prop-2- enoyl}piperazin-1-yl) nicotinonitrile |
| 14.6 | 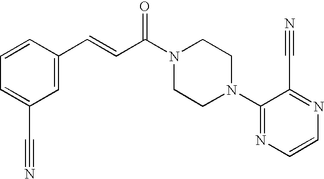 | 3-{4-[(2E)-3-(3- cyanophenyl)prop-2- enoyl]piperazin-1- yl}pyrazine-2-carbonitrile |
| 14.7 | 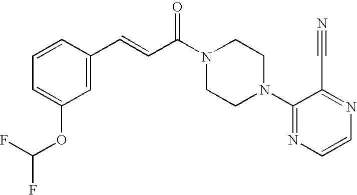 | 3-(4-{(2E)-3-[3- (difluoromethoxy) phenyl]prop-2- enoyl}piperazin-1-yl) pyrazine-2-carbonitrile |
| 14.8 | 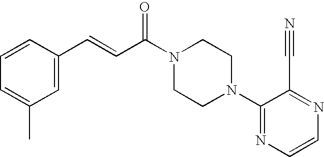 | 3-{4-[(2E)-3-(3- methylphenyl)prop-2- enoyl]piperazin-1- yl}pyrazine-2-carbonitrile |
| 15.1 | 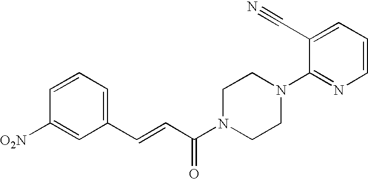 | 2-{4-[(2E)-3-(3- nitrophenyl)prop-2- enoyl]piperazin-1- yl}nicotinonitrile |
| 15.2 | 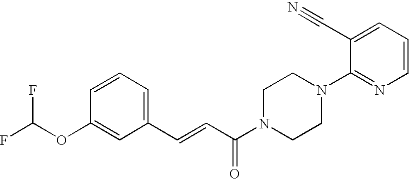 | 2-(4-{(2E)-3-[3- (difluoromethoxy) phenyl]prop-2- enoyl}piperazin-1-yl) a nicotinonitrile |
| 15.3 | 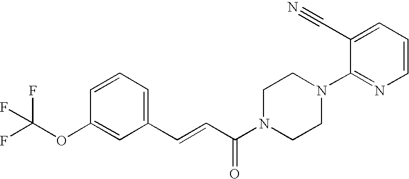 | 2-(4-{(2E)-3-[3- (trifluoromethoxy) phenyl]prop-2- enoyl}piperazin-1-yl) nicotinonitrile |
| 15.4 | 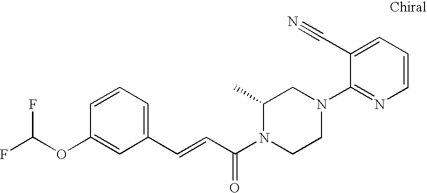 | 2-((3R)-4-{(2E)-3-[3- (difluoro- methoxy)phenyl]prop-2- enoyl}-3-methylpiperazin-1- yl)nicotinonitrile |
| 15.5 | 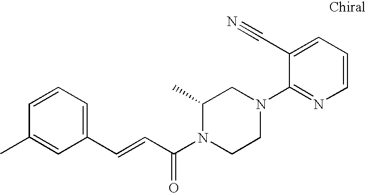 | 2-{(3R)-3-methyl-4-[(2E)-3- (3-methylphenyl)prop-2- enoyl]piperazin-1- yl}nicotinonitrile |
| 15.6 | 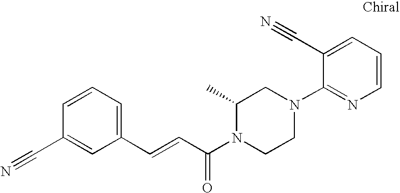 | 2-{(3R)-4-[(2E)-3-(3- cyanophenyl) prop-2-enoyl]- 3-methylpiperazin-1- yl}nicotinonitrile |
| 16.1 | 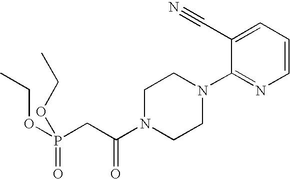 | Diethyl {2-[4-(3- cyanopyridin-2-yl) piperazin-1-yl]-2- oxoethyl}phosphonate |
| 17.1 | 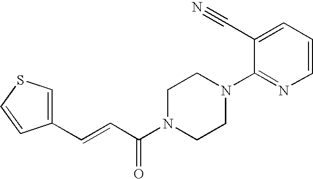 | 2-{4-[(2E)-3-(3- thienyl)prop-2- enoyl]piperazin-1- yl}nicotinonitrile |
| 17.2 | 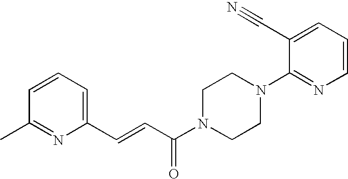 | 2-{4-[(2E)-3-(6- methylpyridin-2-yl)prop-2- enoyl]piperazin-1-yl}nicotinonitrile |
| 17.3 | 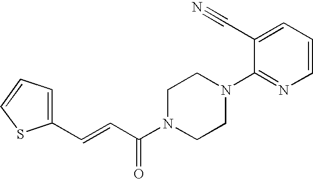 | 2-{4-[(2E)-3-(2- thienyl)prop-2- enoyl]piperazin-1- yl}nicotinonitrile |
| 19.1 | 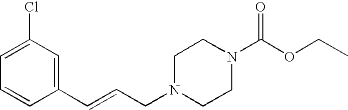 | Ethyl 4-[(2E)-3-(3-chloro- phenyl)prop-2-en-1- yl]piperazine-1-carboxylate |
| 19.2 | 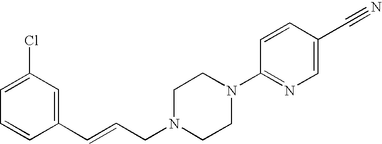 | 6-{4-[(2E)-3-(3- chlorophenyl)prop-2-en-1- yl]piperazin-1-yl}nicotinonitrile |
| 23.1 | 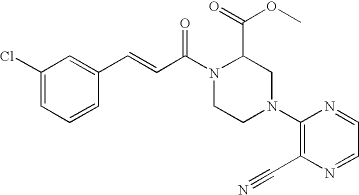 | 4-[3-(3-Chloro-phenyl)- acryloyl]-3′-cyano-3,4,5,6- tetrahydro-2H- [1,2]bipyrazinyl-3- carboxylic acid methyl ester |
| 24.1 | 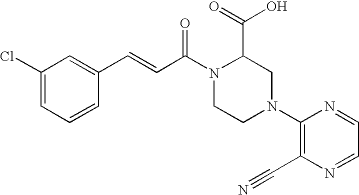 | 4-[3-(3-Chloro-phenyl)- acryloyl]-3′-cyano-3,4,5,6- tetrahydro-2H- [1,2′]bipyrazinyl-3- carboxylic acid |
| 26.1 | 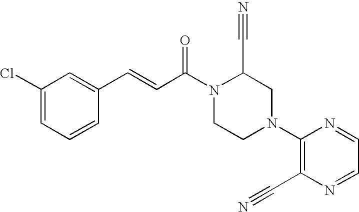 | 4-[3-(3-Chloro-phenyl)- acryloyl]-3,4,5,6-tetrhydro- 2H-[1,2′]bipyrazinyl-3,3′- dicarbonitrile |
| 27.1 | 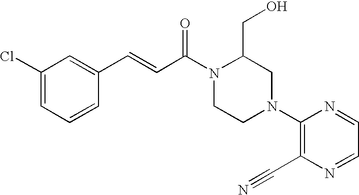 | 4-[3-(3-Chloro-phenyl)- acryloyl]-3-hydroxymethyl- 3,4,5,6-tetrahydro-2H- [1,2′]bipyrazinyl-3′- carbonitrile |
| 28.1 | 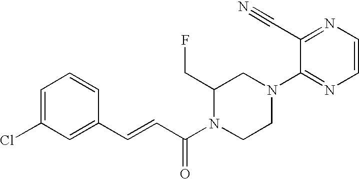 | 3-[4-[(2E)-3-(3- chlorophenyl)prop-2-enoyl]- 3-(fluoromethyl)piperazin-1- yl]pyrazine-2-carbonitrile |
| 29.1 | 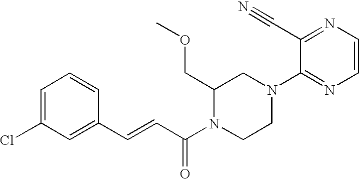 | 3-[4-[(2E)-3-(3- chlorophenyl)prop-2-enoyl]- 3-(methoxymethyl)piperazin- 1-yl]pyrazine-2-carbonitrile |

Pharmaceutical Composition

The compounds of the present invention may be formulated into conventional pharmaceutical composition comprising a compound of formula I, or a pharmaceutically acceptable salt or solvate thereof, in association with a pharmaceutically acceptable carrier or excipient. The pharmaceutically acceptable carriers can be either solid or liquid. Solid form preparations include, but are not limited to, powders, tablets, dispersible granules, capsules, cachets, and suppositories.

A solid carrier can be one or more substances, which may also act as diluents, flavoring agents, solubilizers, lubricants, suspending agents, binders, or tablet disintegrating agents. A solid carrier can also be an encapsulating material.

In powders, the carrier is a finely divided solid, which is in a mixture with the finely divided compound of the invention, or the active component. In tablets, the active component is mixed with the carrier having the necessary binding properties in suitable proportions and compacted in the shape and size desired.

For preparing suppository compositions, a low-melting wax such as a mixture of fatty acid glycerides and cocoa butter is first melted and the active ingredient is dispersed therein by, for example, stirring. The molten homogeneous mixture is then poured into convenient sized moulds and allowed to cool and solidify.

Suitable carriers include, but are not limited to, magnesium carbonate, magnesium stearate, talc, lactose, sugar, pectin, dextrin, starch, tragacanth, methyl cellulose, sodium carboxymethyl cellulose, low-melting wax, cocoa butter, and the like.

The term composition is also intended to include the formulation of the active component with encapsulating material as a carrier providing a capsule in which the active component (with or without other carriers) is surrounded by a carrier which is thus in association with it. Similarly, cachets are included.

Tablets, powders, cachets, and capsules can be used as solid dosage forms suitable for oral administration.

Liquid form compositions include solutions, suspensions, and emulsions. For example, sterile water or water propylene glycol solutions of the active compounds may be liquid preparations suitable for parenteral administration. Liquid compositions can also be formulated in solution in aqueous polyethylene glycol solution.

Aqueous solutions for oral administration can be prepared by dissolving the active component in water and adding suitable colorants, flavoring agents, stabilizers, and thickening agents as desired. Aqueous suspensions for oral use can be made by dispersing the finely divided active component in water together with a viscous material such as natural synthetic gums, resins, methyl cellulose, sodium carboxymethyl cellulose, and other suspending agents known to the pharmaceutical formulation art. Exemplary compositions intended for oral use may contain one or more coloring, sweetening, flavoring and/or preservative agents.

Depending on the mode of administration, the pharmaceutical composition will include from about 0.05% w (percent by weight) to about 99% w, more particularly, from about 0.10% w to 50% w, of the compound of the invention, all percentages by weight being based on the total weight of the composition.

A therapeutically effective amount for the practice of the present invention can be determined by one of ordinary skill in the art using known criteria including the age, weight and response of the individual patient, and interpreted within the context of the disease which is being treated or which is being prevented.

Medical Use

It has been found that the compounds according to the present invention, exhibit a high degree of potency and selectivity for individual metabotropic glutamate receptor (mGluR) subtypes. Accordingly, the compounds of the present invention are expected to be useful in the treatment of conditions associated with excitatory activation of mGluR5 and for inhibiting neuronal damage caused by excitatory activation of mGluR5. The compounds may be used to produce an inhibitory effect of mGluR5 in mammals, including man.

The Group I mGluR receptors including mGluR5 are highly expressed in the central and peripheral nervous system and in other tissues. Thus, it is expected that the compounds of the invention are well suited for the treatment of mGluR5-mediated disorders such as acute and chronic neurological and psychiatric disorders, gastrointestinal disorders, and chronic and acute pain disorders.

The invention relates to compounds of Formula I, as defined hereinbefore, for use in therapy.

The invention relates to compounds of Formula I, as defined hereinbefore, for use in treatment of mGluR5-mediated disorders.

The invention relates to compounds of Formula I, as defined hereinbefore, for use in treatment of Alzheimer's disease senile dementia, AIDS-induced dementia, Parkinson's disease, amylotropic lateral sclerosis, Huntington's Chorea, migraine, epilepsy, schizophrenia, depression, anxiety, acute anxiety, ophthalmological disorders such as retinopathies, diabetic retinopathies, glaucoma, auditory neuropathic disorders such as tinnitus, chemotherapy induced neuropathies, post-herpetic neuralgia and trigeminal neuralgia, tolerance, dependency, Fragile X, autism, mental retardation, schizophrenia and Down's Syndrome.

The invention relates to compounds of Formula I, as defined above, for use in treatment of pain related to migraine, inflammatory pain, neuropathic pain disorders such as diabetic neuropathies, arthritis and rheumatoid diseases, low back pain, post-operative pain and pain associated with various conditions including angina, renal or billiary colic, menstruation, migraine and gout.

The invention relates to compounds of Formula I as defined hereinbefore, for use in treatment of stroke, head trauma, anoxic and ischemic injuries, hypoglycemia, cardiovascular diseases and epilepsy.

The invention relates to compounds of Formula I as defined hereinbefore, for use in treatment of appetite-related disorders.

The invention relates to compounds of Formula I as defined hereinbefore, for use in treatment of cravings or addiction.

The present invention relates also to the use of a compound of Formula I as defined hereinbefore, in the manufacture of a medicament for the treatment of mGluR Group I receptor-mediated disorders and any disorder listed above.

One embodiment of the invention relates to the use of a compound according to Formula I in the treatment of gastrointestinal disorders.

Another embodiment of the invention relates to the use of a Formula I compound for the manufacture of a medicament for inhibition of transient lower esophageal sphincter relaxations, for the treatment of GERD, for the prevention of G.I. reflux, for the treatment regurgitation, for treatment of asthma, for treatment of laryngitis, for treatment of lung disease, and for the management of failure to thrive.

Another embodiment of the invention relates to the use of a Formula I compound for the manufacture of a medicament for the treatment or prevention of obesity or overweight, (e.g., promotion of weight loss and maintenance of weight loss), prevention or reversal of weight gain (e.g., rebound, medication-induced or subsequent to cessation of smoking), for modulation of appetite and/or satiety, eating disorders (e.g. binge eating, anorexia, bulimia and compulsive)

A further embodiment of the invention relates to the use of a Formula I compound for the manufacture of a medicament for the treatment of cravings or addictions (such as for drugs, tobacco, alcohol, any appetizing macronutrients or non-essential food items).

The invention also provides a method of treatment of mGluR5-mediated disorders and any disorder listed above, in a patient suffering from, or at risk of, said condition, which comprises administering to the patient an effective amount of a compound of Formula I, as hereinbefore defined.

The dose required for the therapeutic or preventive treatment of a particular disorder will necessarily be varied depending on the host treated, the route of administration and the severity of the illness being treated.

In the context of the present specification, the term “therapy” and “treatment” includes prevention or prophylaxis, unless there are specific indications to the contrary. The terms “therapeutic” and “therapeutically” should be construed accordingly.

In this specification, unless stated otherwise, the term “antagonist” and “inhibitor” shall mean a compound that by any means, partly or completely, blocks the transduction pathway leading to the production of a response by the ligand.

The term “disorder”, unless stated otherwise, means any condition and disease associated with metabotropic glutamate receptor activity.

Non-Medical Use

In addition to their use in therapeutic medicine, the compounds of Formula I, as well as salts and hydrates of such compounds, are useful as pharmacological tools in the development and standardization of in vitro and in vivo test systems for the evaluation of the effects of inhibitors of mGluR related activity in laboratory animals such as cats, dogs, rabbits, monkeys, rats and mice, as part of the search for new therapeutics agents.

Process of Preparation

Another aspect of the present invention provides processes for preparing compounds of Formula I, or salts or hydrates thereof. Processes for the preparation of the compounds in the present invention are described herein.

Throughout the following description of such processes it is to be understood that, where appropriate, suitable protecting groups will be added to, and subsequently removed from, the various reactants and intermediates in a manner that will be readily understood by one skilled in the art of organic synthesis. Conventional procedures for using such protecting groups as well as examples of suitable protecting groups are described, for example, in “Protective Groups in Organic Synthesis”, T. W. Green, P. G. M. Wuts, Wiley-Interscience, New York, (1999). It also is to be understood that a transformation of a group or substituent into another group or substituent by chemical manipulation can be conducted on any intermediate or final product on the synthetic path toward the final product, in which the possible type of transformation is limited only by inherent incompatibility of other functionalities carried by the molecule at that stage to the conditions or reagents employed in the transformation. Such inherent incompatibilities, and ways to circumvent them by carrying out appropriate transformations and synthetic steps in a suitable order, will be readily understood to the one skilled in the art of organic synthesis. Examples of transformations are given below, and it is to be understood that the described transformations are not limited only to the generic groups or substituents for which the transformations are exemplified. References and descriptions on other suitable transformations are given in “Comprehensive Organic Transformations—A Guide to Functional Group Preparations” R. C. Larock, VHC Publishers, Inc. (1989). References and descriptions of other suitable reactions are described in textbooks of organic chemistry, for example, “Advanced Organic Chemistry”, March, 4th ed. McGraw Hill (1992) or, “Organic Synthesis”, Smith, McGraw Hill, (1994).

Techniques for purification of intermediates and final products include for example, normal and reversed phase chromatography on column or rotating plate, recrystallisation, distillation and liquid-liquid or solid-liquid extraction, which will be readily understood by the one skilled in the art. The definitions of substituents and groups are as in formula I except where defined differently. The term “room temperature” and “ambient temperature” shall mean, unless otherwise specified, a temperature between 16 and 25° C.

For the preparation of compounds of Formula I wherein R² is Ar and A is C═O, the cinnamic acid or cinnamoyl amide may be prepared in several ways. One such method uses a Wittig type olefination from an aryl aldehyde and a dialkyl phosphonate, with a base such as sodium hydride to generate the anion in a solvent such as THF, with heating at reflux to effect the olefination.

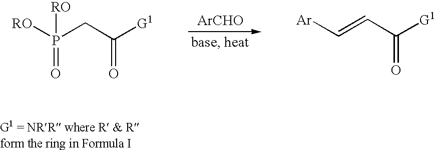

An alternative method for the preparation of compounds of Formula I uses an aryl bromide or iodide with an α,β-unsaturated acid or amide using palladium catalysts such as Pd(OAc)₂ and a base such as triethylamine in a solvent such as acetonitrile or dimethylacetamide. Salts such as sodium acetate and tetrabutylammonium chloride may be employed, as well phosphine ligands may be used in some cases to facilitate the reaction. The reaction is typically carried out with heating between 80° C. and 180° C., either conventionally or using microwave irradiation.

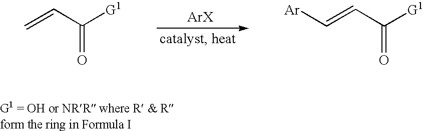

When G₁ is OH, the acid may be coupled to an amine such as a piperazine using standard conditions such as EDCI, TBTU to activate the acid with triethylamine, DMAP or HOBt, in a solvent such as DMF or dichloromethane. Alternatively the acid may be activated as the acid chloride using oxalyl chloride in dichloromethane with triethylamine and a few drops of DMF.

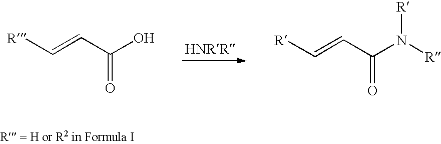

For the preparation of compounds of Formula I wherein R² is Ar and A is CHR⁴, a 3-component coupling between an amine, a vinyl boronic acid and an aldehyde may be carried out in a solvent such as 1,4-dioxane at elevated temperature (90° C.).

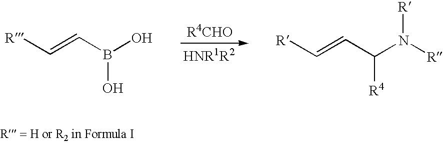

In compounds of Formula I wherein X is N and R³ is a heteroaryl, R³ may be introduced from on the free amine using a heteroaryl chloride in a solvent such as acetonitrile or DMF, at ambient temperatures or with heating between 80 to 100° C. conventionally or with microwave irradiation at temperatures up to 240° C. When the aryl group is less activated, catalysts may be employed. This may be done at an early stage using a protected bisamine such as piperazine, or it may be introduced at a late stage.

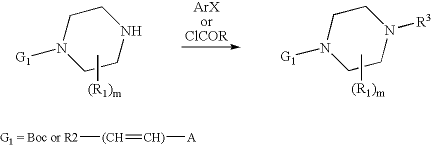

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

General Methods

All starting materials are commercially available or earlier described in the literature.

The ¹H and ¹³C NMR spectra were recorded either on Bruker 300, Bruker DPX400 or Varian +400 spectrometers operating at 300, 400 and 400 MHz for ¹H NMR respectively, using TMS or the residual solvent signal as reference, in deuterated chloroform as solvent unless otherwise indicated. All reported chemical shifts are in ppm on the delta-scale, and the fine splitting of the signals as appearing in the recordings (s: singlet, br s: broad singlet, d: doublet, t: triplet, q: quartet, m: multiplet). Unless otherwise indicated, in the tables below ¹H NMR data was obtained at 300 MHz, using CDCl₃ as the solvent.

Analytical in line liquid chromatography separations followed by mass spectra detections, were recorded on a Waters LCMS consisting of an Alliance 2795 (LC) and a ZQ single quadropole mass spectrometer. The mass spectrometer was equipped with an electrospray ion source operated in a positive and/or negative ion mode. The ion spray voltage was ±3 kV and the mass spectrometer was scanned from m/z 100-700 at a scan time of 0.8 s. To the column, X-Terra MS, Waters, C8, 2.1×50 mm, 3.5 mm, was applied a linear gradient from 5% to 100% acetonitrile in 10 mM ammonium acetate (aq.), or in 0.1% TFA (aq.).

Preparative reversed phase chromatography was run on a Gilson autopreparative HPLC with a diode array detector using an XTerra MS C8, 19×300 mm, 7 mm as column.

Purification by a chromatotron was performed on rotating silica gel/gypsum (Merck, 60 PF-254 with calcium sulphate) coated glass sheets, with coating layer of 1, 2, or 4 mm using a TC Research 7924T chromatotron.

Purification of products were also done using Chem Elut Extraction Columns (Varian, cat #1219-8002), Mega BE-SI (Bond Elut Silica) SPE Columns (Varian, cat # 12256018; 12256026; 12256034), or by flash chromatography in silica-filled glass columns.

Microwave heating was performed in an Emrys Optimizer from Biotage/Personal Chemistry or a Smith Synthesizer Single-mode microwave cavity producing continuous irradiation at 2450 MHz (Personal Chemistry AB, Uppsala, Sweden).

The pharmacological properties of the compounds of the invention can be analyzed using standard assays for functional activity. Examples of glutamate receptor assays are well known in the art as described in for example Aramori et al., Neuron 8:757 (1992), Tanabe et al., Neuron 8:169 (1992), Miller et al., J. Neuroscience 15: 6103 (1995), Balazs, et al., J. Neurochemistry 69:151 (1997). The methodology described in these publications is incorporated herein by reference. Conveniently, the compounds of the invention can be studied by means of an assay that measures the mobilization of intracellular calcium, [Ca²⁺]ᵢ in cells expressing mGluR5.

Intracellular calcium mobilization was measured by detecting changes in fluorescence of cells loaded with the fluorescent indicator fluo-3. Fluorescent signals were measured using the FLIPR system (Molecular Devices). A two addition experiment was used that could detect compounds that either activate or antagonize the receptor.

For FLIPR analysis, cells expressing human mGluR5d were seeded on collagen coated clear bottom 96-well plates with black sides and analysis of [Ca²⁺]ᵢ mobilization was done 24 hours after seeding.

FLIPR experiments were done using a laser setting of 0.800 W and a 0.4 second CCD camera shutter speed. Each FLIPR experiment was initiated with 160 μL of buffer present in each well of the cell plate. After each addition of the compound, the fluorescence signal was sampled 50 times at 1 second intervals followed by 3 samples at 5 second intervals. Responses were measured as the peak height of the response within the sample period.

EC₅₀ and IC₅₀ determinations were made from data obtained from 8-point concentration response curves (CRC) performed in duplicate. Agonist CRC were generated by scaling all responses to the maximal response observed for the plate. Antagonist block of the agonist challenge was normalized to the average response of the agonist challenge in 14 control wells on the same plate.

We have validated a secondary functional assay for mGluR5d based on Inositol Phosphate (IP₃) turnover. IP₃ accumulation is measured as an index of receptor mediated phospholipase C turnover. GHEK cells stably expressing the human mGluR5d receptors were incubated with [3H] myo-inositol overnight, washed three times in HEPES buffered saline and pre-incubated for 10 minutes with 10 mM LiCl. Compounds (agonists) were added and incubated for 30 minutes at 37° C. Antagonist activity was determined by pre-incubating test compounds for 15 minutes, then incubating in the presence of glutamate (80 μM) or DHPG (30 μM) for 30 minutes. Reactions were terminated by the addition of perchloric acid (5%). Samples were collected and neutralized, and inositol phosphates were separated using Gravity-Fed Ion-Exchange Columns.

Abbreviations

BOC tert-butoxycarbonyl
BSA Bovine Serum Albumin
CCD Charge Coupled Device
CRC Concentration Response Curve
DBU 1,8-diazabicyclo[5.4.0]undec-7-ene
DCM dichloromethane
DHPG 3,5-dihydroxyphenylglycine;
DIBAL diisobutylaluminum hydride
DMF N,N-dimethylformamide
DMSO dimethyl sulfoxide
EDTA Ethylene Diamine Tetraacetic Acid
Et₃N triethylamine
FLIPR Fluorometric Imaging Plate reader
GC/MS gas chromatograph coupled mass spectroscopy
GHEK Human Embryonic Kidney expressing Glutamate Transporter
HEPES 4-(2-hydroxyethyl)-1-piperazineethanesulfonic acid (buffer)
IP₃ inositol triphosphate
MCPBA 3-chloroperbenzoic acid
MeOH methanol
NMP N-Methylpyrrolidinone
NMR nuclear magnetic resonance
PCC pyridinium chlorochromate
ppm parts per million
RT room temperature
SPE solid phase extraction
TFA trifluoroacetic acid
THF tetrahydrofuran
TLC thin layer chromatography

### EXAMPLE 1.1

### 2-Chloro-5-fluoronicotinonitrile

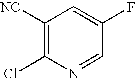

Oxalyl chloride (2M in dichloromethane, 6 mL, 12 mmol) was added to a solution of 2-chloro-5-fluoronicotinic acid (1 g, 5.7 mmol) in dichloromethane (15 mL), followed by a few drops of DMF. The reaction mixture was stirred at room temperature for 2 h. Excess oxalyl chloride and solvent were removed in vacuo to give the acid chloride.

The residue was taken up in THF (10 mL) and cooled to 0° C. prior to the addition of concentrated aqueous ammonia (30%, 3 mL). After 15 min, TLC confirmed the reaction was complete. The product was extracted into diethyl ether, and the organic layer was dried and evaporated in vacuo to give 2-chloro-5-fluoronicotinamide (white solid, 945 mg, 95%).

A solution of 2-chloro-5-fluoronicotinamide (350 mg, 2 mmol) and 2,4,6-trichloro-1,3,5-triazine (220 mg, 1.2 mmol) in DMF (1 mL) was stirred at room temperature. After 5 minutes a slight exotherm was noted. The reaction mixture was stirred for an additional 20 min prior to quenching with water and extraction with diethyl ether. The organic layer was dried and evaporated in vacuo to give the title compound (354 mg, 88% calculated yield based on DMF 22% by wt., used without further purification). ¹H NMR (300 MHz, CDCl₃): δ (ppm)=8.48 (dd, 1H); 7.80 (dd, 1H).

### EXAMPLE 2.1

### di-tert-butyl 2-cyanopiperazine-1,4-dicarboxylate

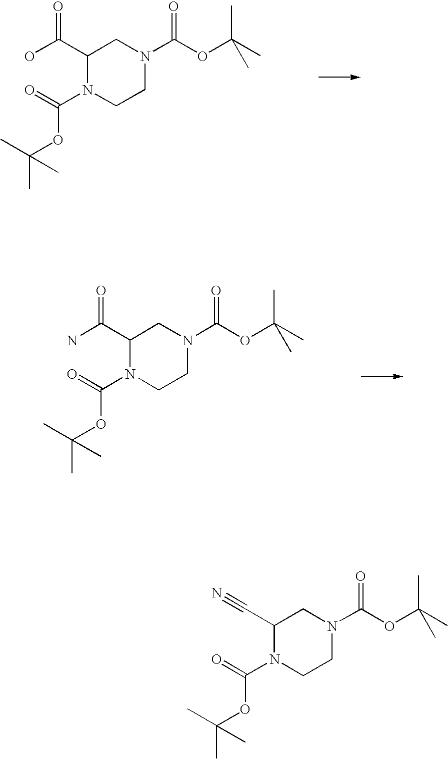

Isobutyl chloroformate (3.42 mL, 26.1 mmol) was added to a cold (0° C.) solution of 1,4-bis(tert-butoxycarbonyl)piperazine-2-carboxylic acid (8.21 g, 24.85 mmol) and triethylamine (13.8 mL, 26.1 mmol) in THF (50 mL) and DMF (20 mL). The reaction mixture was stirred −5-0° C. for 1 h. Concentrated aqueous ammonia (28%, 20 mL) was added and the reaction mixture was stirred for 1 h. The product was extracted into ethyl acetate. The organic layer was washed with water (3×) and brine (1×), dried over magnesium sulfate and evaporated in vacuo to give di-tert-butyl 2-(aminocarbonyl)piperazine-1,4-dicarboxylate (white solid, 6.33 g, 77.3%) which was used without further purification.

2,4,6-Trichloro-1,3,5-triazine (2.12 g, 11.52 mmol) and DMF (15 mL) were added and the resulting mixture was stirred at room temperature for 1 h prior to quenching with water and extraction with ethyl acetate. The organic layer was washed with water (3×) and brine (1×), dried over magnesium sulfate, and evaporated in vacuo to give the title compound (5.36 g, 89.5%). ¹H NMR (300 MHz, CDCl₃): δ (ppm)=5.08 (br m, 1H); 4.36 (br m, 1H); 4.25 (br m, 1H); 3.95 (br d, 1H); 3.06 (br t, 2H); 2.80 (br m, 1H); 1.51 (s, 18H).

### EXAMPLE 3.1

### di(tert-Butoxycarbonyl)-2-(1-methyl-1H-tetrazol-5-yl)piperazine-1,4-dicarboxylate & di(tert-Butoxycarbonyl)-2-(2-methyl-2H-tetrazol-5-yl)piperazine-1,4-dicarboxylate

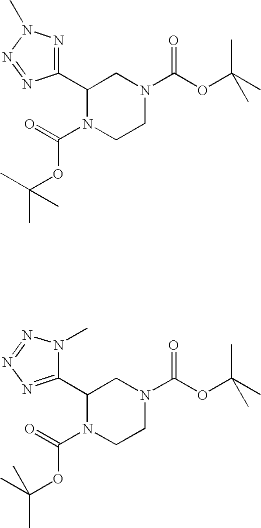

A mixture of di-tert-butyl 2-cyanopiperazine-1,4-dicarboxylate (3.11 g, 10 mmol), sodium azide (715 mg, 11 mmol), ammonium chloride (588 mg, 11 mmol) in DMF (8 mL) was heated at 100° C. overnight. The reaction was cooled to 0° C. and iodomethane (0.68 mL, 11 mmol) was added. The reaction mixture was stirred at 0° C. for 3 h prior to quenching with water. The resulting white solid was collected by filtration, washed with water and dissolved in ethyl acetate, dried over magnesium sulfate, filtered and evaporated in vacuo. Chromatography (silica gel, 5-25% ethyl acetate in hexanes) gave the title compounds. 73.1 (1.68 g, 45.6%); ¹H NMR (300 MHz, CDCl₃): δ (ppm)=5.52 (br m, 1H); 4.60 (br d, 1H); 4.32 (s, 3H); 4.08 (br m, 1H); 3.95 (br d, 1H); 3.33 (br m, 2H); 2.90 (br m, 1H); 1.46 (br s, 9H); 1.38 (br s, 9H). 73.2 (1.035 g, 28.6%); ¹H NMR (300 MHz, CDCl₃): δ (ppm)=5.50 (br m, 1H); 4.30 (br d, 1H); 4.12 (br s, 3H); 4.05 (br m, 1H); 3.95 (br d, 1H); 3.45 (br m, 2H); 2.95-3.15(br m, 1H); 1.46(br s, 9H); 1.41(s, 9H).

### EXAMPLE 4.1

### 2-(2-methyl-2H-tetrazol-5-yl)piperazine bis(trifluoroacetate)

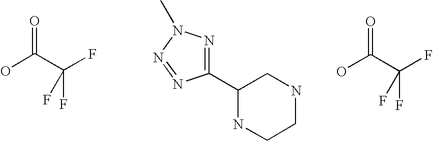

A solution of di(tert-butoxycarbonyl)-2-(2-methyl-2H-tetrazol-5-yl)piperazine-1-carboxylic acid (368.4 mg, 1.0 mmol) and trifluoroacetic acid (2 mL) in dichloromethane (1 mL) was stiffed at 0° C. for 1 h. Excess trifluoroacetic acid was removed in vacuo, and the resulting product was triturated with diethyl ether to provide the title compound (white solid, 350 mg, 88.3%). ¹H NMR (300 MHz, DMSO-d₆): δ (ppm)=4.88 (dd, 1H); 4.43 (s, 3H); 3.79 (dd, 1H); 3.46 (d, 2H); 3.1-3.4 (complex m's, 3H).

### EXAMPLE 5.1

### 2-(1-methyl-1H-tetrazol-5-yl)piperazine Dihydrochloride

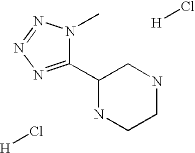

A solution of di(tert-butoxycarbonyl)-2-(1-methyl-1H-tetrazol-5-yl)piperazine-1,4-dicarboxylic acid (368.4 mg, 1.0 mmol) and trifluoroacetic acid (2 mL) in dichloromethane (1 mL) was stirred at 0° C. for 1 h. Excess trifluoroacetic acid was removed in vacuo. The resulting product was triturated with diethyl ether and ethyl acetate, then dissolved in ethanol (1.5 mL). HCl (6N aqueous, 1.5 mL) was added and the resulting mixture was refluxed for 4 h. Removal of the solvent in vacuo and trituration with diethyl ether yielded the title compound (white solid, 180 mg, 74.6%). ¹H NMR (300 MHz, DMSO-d₆): δ (ppm)=5.28 (d, 1H); 4.19 (s, 3H); 3.79 (d, 1H); 3.2-3.6 (complex m's, 5H).

### EXAMPLE 6

### General Procedure: Nucleophilic Displacement with 2-Chloro-3-NitroPyridine

Piperazine (2-5 mmol) and 2-chloro-3-nitro-pyridine (1 mmol) were dissolved in DMF or acetonitrile (2-3 mL) and stirred for 5 min at room temperature. A slight exotherm was observed shortly after addition of the solvent. When TLC analysis showed that the reaction was complete, the mixture was diluted with dichloromethane, and washed with water. The organic layer was dried, filtered and concentrated, then chromatographed in 10% methanol in dichloromethane to yield the desired product.

In this manner the following compounds were synthesized:

| Example | Structure | Name | Yield |
|---|---|---|---|
| 6.1 | 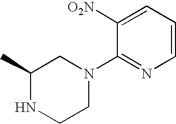 | (3S)-3-methyl-1-(3-nitropyridin-2-yl) piperazine | 92% |
| ¹H | 1.11(d, J=6.3 Hz, 3H); 2.74(dd, J=12.8, 10.4 Hz, 1H); 2.99(m, 4H); 3.72(m, 2H); | | |
| NMR | 6.74(dd, J=8.1, 4.5 Hz, 1H); 8.14(dd, J=8.1, 1.8 Hz, 1H); 8.34(dd, J=4.5, 1.8 Hz, | | |
| | 1H). | | |
| 6.2 | 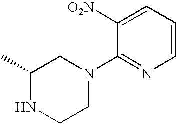 | (3R)-3-methyl-1-(3-nitropyridin-2-yl) piperazine | 100% |
| ¹H | 1.11(d, J=6.3 Hz, 3H); 2.74(dd, J=12.8, 10.4 Hz, 1H); 2.99(m, 4H); 3.72(m, 2H); | | |
| NMR | 6 74(dd, J=8.1, 4.5 Hz, 1H); 8.14(dd, J=8.1, 1.8 Hz, 1H); 8.34(dd, J=4.5, 1.8 Hz, | | |
| | 1H). | | |
| 6.3 | 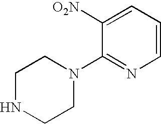 | 1-(3-nitropyridin-2-yl)piperazine | 63% |

| | |
|---|---|
| ¹H | 3.00(m, 4H); 3.45(m, 4H); 6.75(dd, J=8.1, 4.5 Hz, 1H); 8.14(dd, J=8.1, 1.8 Hz, 1H); |
| NMR | 8.34(dd, J=4.5, 1.8 Hz, 1H). |

### EXAMPLE 7

### General Procedure: Thermal Coupling to Heteroaryl Chloride

A solution of 2-chloro-5-fluoronicotinonitrile (1 mmol) and amine (1.5 mmol) in 10 ml of acetonitrile was heated for 30 min at 85-90° C. in a screw cap vial, then diluted with DCM, washed with water and dried over anhydrous sodium sulfate. SPE column chromatography (silica gel, 0-10% MeOH in DCM) gave the desired product. For methyl-tetrazole piperazine salts, the combination of pyrazine (1.05 mmol)/amine (1 mmol)/Et₃N (4 mmol) was used to reduce the amount of piperazine and to neutralize salts in situ.

| Example | Structure | Name | Yield |
|---|---|---|---|
| 7.1 | 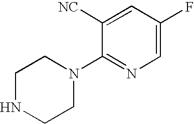 | 5-fluoro-2-piperazin-1-yl-solid, nicotinonitrile | It yellow solid, 217 mg, 33% |
| ¹H | 8.25(m, 1H); 7.54(m, 1H); 4.09(m, 1H); 3.48(m, 1H); 3.01(m, 4H); 1.15(m, 3H) | | |
| NMR | | | |
| 7.2 | 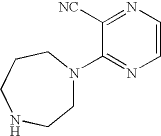 | 3-(1,4-diazepan-1-yl)pyrazine-2- carbonitrile | yellow oil, 390.8 mg, 54% |
| ¹H | 8.22(m, 1H); 7.95(m, 1H); 3.95(m, 4H); 3.12(m, 2H); 2.90(m, 2H); 2.00(m, 3H) | | |
| NMR | | | |
| 7.3 | 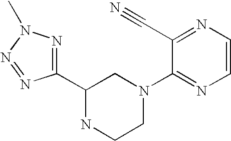 | 3-[3-(2-methyl-2H-tetrazol-5-yl) piperazin-1-yl]pyrazine-2-carbonitrile | sticky oil 130 mg, 95.8% |
| ¹H | 8.30(d, 1H); 8.08(d, 1H); 4.69(dm, 1H); 4.40(dm, 1H); 4.36(s, 3H); 4.34(m, 1H); 3.54 | | |
| NMR | (dd td, 2H); 3.29(dt, 1H); 3.15(td, 1H) | | |
| 7.4 | 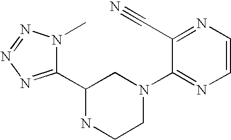 | 3-[3-(1-methyl-1H-tetrazol-5-yl) piperazin-1-yl]pyrazine-2-carbonitrile | Sticky oil 125 mg, 92.1% |

| | |
|---|---|
| ¹H | 8.32(d, 1H); 8.13(d, 1H); 4.58(dm, 1H); 4.37(dd, 1H); 4.34(dm, 1H); 4.23(s, 3H); |
| NMR | 3.53(dd, 1H); 3.25-3.4(td dt, 2H); 3.13(tm, 1H) |

### EXAMPLE 8

### General Procedure: Microwave assisted Nucleophilic displacement of 2-Chloropyridine

Piperazine (5 mmol) and 2,3 dichloropyridine (1 mmol) were dissolved in dimethylformamide (1 mL) in a microwave safe vial. The vial was sealed and the reaction was heated to 240° C. via a microwave for 5 min. The reaction mixture was then diluted with dichloromethane and washed with water. The organic phase was then washed a second time with water, then dried (Na₂SO₄), filtered and concentrated under reduced pressure. The crude product was then purified via flash chromatography with 10% methanol in dichloromethane to yield the desired product.

In this manner the following compound was synthesized:

| Example | Structure | Name | Yield |
|---|---|---|---|
| 8.1 | 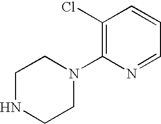 | 1-(3-Chloro-pyridin-2-yl)-piperazine | 39% |

| | |
|---|---|
| ¹H | 1.65(s, broad, 1H); 3.07(m, 4H); 3.33(m, 4H); 6.85(m, 1H); 7.60(dd, J=7.8, 1.8 Hz, |
| NMR | 1H); 8.21(m, 1H). |

### EXAMPLE 9

### General Procedure: Palladium Catalyzed Coupling to Heteroaryl Chloride

2-Chloro-nicotinonitrile (1.0 mmol), (S)-2-methyl piperazine (1.5 mmol), sodium tert-butoxide (1.5 mmol) and tris(dibenzylideneacetone)-dipalladium(0) (0.04 mmol) were added to a screw cap vial. 2,8,9-Triisobutyl-2,5,8,9-tetraaza-1-phospha-bicyclo[3.3.3]undecane (0.08 mmol) was dissolved in toluene (5 mL) and this solution was added to the other reagents. The reaction mixture was stirred at 100° C. overnight. The solution was diluted with dichloromethane and washed with water. The organic phase was dried, filtered and concentrated, then purified by flash chromatography in 10% (2M ammonia in methanol) in dichloromethane to yield the desired product.

In this manner the following compounds were synthesized:

| Example | Structure | Name | Yield |
|---|---|---|---|
| 9.1 | 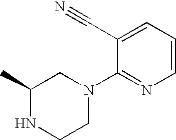 | 2-[(3S)-3-methylpiperazin-1-yl]nicotinonitrile | 64% |
| ¹H | 1.03(d, J=6.3 Hz, 3H); 1.73(s, broad, 1H); 2.59(dd, J=12.9, 10.2 Hz, 1H); 2.94(m, | | |
| NMR | 4H); 4.16(m, 2H); 6.64(dd, J=7.8, 4.8 Hz, 1H); 7.66(dd, J=7.8, 2.1 Hz, 1H); 8.23(dd, | | |
| | J=4.8, 2.1 Hz, 1H). | | |
| 9.2 | 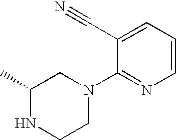 | 2-[(3R)-3-methylpiperazin-1-yl]nicotinonitrile | 46% |
| ¹H | 1.03(d, J=6.3 Hz, 3H); 1.73(s, broad, 1H); 2.59(dd, J=12.9, 10.2 Hz, 1H); 2.94(m, | | |
| NMR | 4H); 4.16(m, 2H); 6.64(dd, J=7.8, 4.8 Hz, 1H); 7.66(dd, J=7.8, 2.1 Hz, 1H); 8.23(dd, | | |
| | J=4.8, 2.1 Hz, 1H). | | |
| 9.3 | 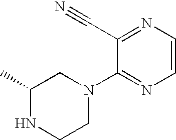 | 3-[(3R)-3-methylpiperazin-1-yl]pyrazine-2-carbonitrile | 66% |

| | |
|---|---|
| ¹H | 1.08(d, J=6.3 Hz, 3H); 2.68(dd, J=12.9, 10.2 Hz, 1H); 2.90(m, 2H); 3.04(m, 2H); |
| NMR | 4.31(m, 2H); 7.92(d, J=2.4 Hz, 1H); 8.18(d, J=2.4 Hz, 1H). |

### EXAMPLE 10.1

### (2E)-3-(3-cyanophenyl)acrylic acid

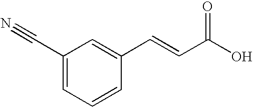

Palladium (II) acetate (13.5 mg, 0.06 mmol), tri (o-tolyl) phosphine (40.2 mg, 0.132 mmol), and triethylamine (0.825 ml, 6.0 mmol) were dissolved in acetonitrile (9 ml) and stirred at room temperature for 15 minutes. 3-Bromo-benzonitrile (1.1 g, 6.0 mmol) was added and the reaction mixture was stirred for an extra 5 minutes at room temperature. Then, acrylic acid was added and the reaction mixture was stirred at 180° C. for 15 minutes in a microwave. The precipitate was filtered off and treated with water to yield a grey solid. The filtrate was concentrated and the residue was treated with water to yield a yellow solid. Each product was separately dissolved in ethanol and dichloromethane, and filtered through celite using ethanol. The filtrate was concentrated in vacuo to yield a light grey solid (552.5 mg, 53.17%). ¹H NMR (300 MHz, DMSO): δ (ppm)=12.59 (s, 1H); 8.24 (m, 1H); 8.06 (m, 1H); 7.87 (m, 1H); 7.62 (m, 2H); 6.71 (m, 1H).

### EXAMPLE 11

### General Procedure: Ethyl-3-aminopropyl carbodiimide hydrochloride (EDCI) Couplings of piperazines with cinnamic acids

Cinnamic acid (1.3 mmol), EDCI (1.3 mmol), dimethylaminopyridine (0.1 mmol) and substituted piperazine (1 mmol) were combined in a screw cap vial and dissolved in dimethylformamide (6 mL). The reaction was stirred overnight at room temperature. The solution was then diluted with dichloromethane and washed with water. The organic phase was dried (Na₂SO₄), filtered and concentrated in vacuo, then chromatographed in 0→50% ethyl acetate in hexanes to yield the desired product.

The following compounds were made in this manner:

| Example | Structure | Name | Yield |
|---|---|---|---|
| 11.1 | 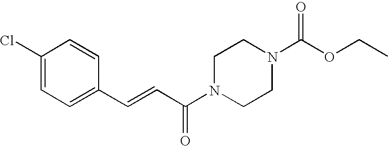 | ethyl 4-[(2E)-3-(4-chloro- phenyl prop-2-enoyl]piperazine-1-carboxylate | 86% |
| ¹H | 1.24(t, J=7.1 Hz, 3H); 3.49(m, 4H); 3.62(m, 2H); 3.65(m, 2H); 6.82(d, J=15.3 Hz, | | |
| NMR | 1H); 7.29(dd, J=8.4, 1.8 Hz, 2H); 7.42(dd, J=8.4, 1.8 Hz, 2H); 7.59(d, J=15.3 Hz, | | |
| | 1H). | | |
| 11.2 | 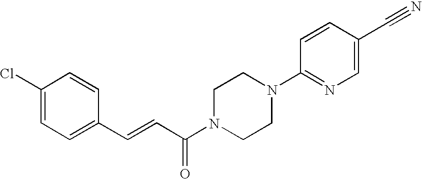 | 6-{4-[(2E)-3-(4-chlorophenyl) prop-2-enoyl]piperazin-1-yl}nicotinonitrile | 98% |
| ¹H | 3.75(m, 2H); 3.86(m, 6H); 6.64(d, J=9Hz, 1H); 6.88(d, J=15.3 Hz, lii); 7.37(d, J= | | |
| NMR | 8 7 Hz, 2H); 7.49(d, J=8.7 Hz, 2H); 7.69(d, J=15.3 Hz, 1H); 7.70(d, J=2.4Hz, 1H); | | |
| | 8.45(m, 1H). | | |
| 11.3 | 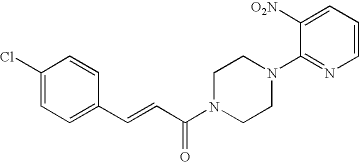 | 1-[(2E)-3-(4-chlorophenyl) prop-2-enoyl]-4-(3-nitro- pyridin-2-yl)piperazine | 53% |
| ¹H | 3.53(m, 4H); 3.84(m, 2H); 3.86(m, 2H); 6.87(d, J=15.3 Hz, 1H); 6.84(m, 1H); 7.34 | | |
| NMR | (dd, J=6.9, 2.1 Hz, 2H); 7.46(dd, J=8.7, 2.1 Hz, 2H); 8.17(dd, J=8.1, 1.8Hz, 1H); | | |
| | 8.37(dd, J=4.5, 1.8 Hz, 1H). | | |
| 11.4 | 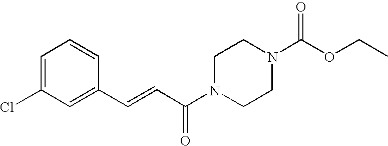 | ethyl 4-[(2E)-3-(3-chloro- phenyl)prop-2-enoyl]piperazine-1-carboxylate | 89% |
| ¹H | 1.21(t, J=7.1 Hz, 3H); 3.47(m, 4H); 3.63(m, 4H); 4.10(q, J=7.1 Hz, 2H); 6.85(d, J= | | |
| NMR | 15.3 Hz, 1H); 7.26(m, 3H); 7.45(m, 1H); 7.54(d, J=15.3 Hz, 1H). | | |
| 11.5 | 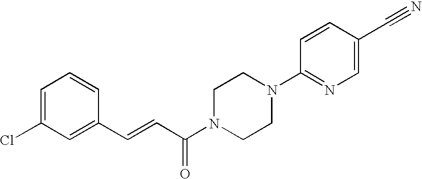 | 6-{4-[(2E)-3-(3-chloro- phenyl)prop-2-enoyl]piperazin-1-yl}nicotinonitrile | 94% |
| ¹H | 3.71(m, 2H); 3.84(m, 6H); 6.62(dd, J=9, 0.6 Hz, 1H); 6.90(d, J=15.3 Hz, 7.30(m, | | |
| NMR | 2H); 7.39(m, 1H); 7.51(m, 1H); 7.64(d, J=15.3 Hz, 1H); 7.64(m, 1H); 8.41(m, 1H). | | |
| 11.6 | 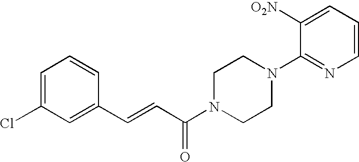 | 1-[(2E)-3-(3-chlorophenyl) prop-2-enoyl]-4-(3-nitro- pyridin-2-yl)piperazine | 48% |
| ¹H | 3.54(m, 4H); 3.85(m, 2H); 3.88(m, 2H); 6.85(m, 1H); 6.90(d, J=15.3 Hz, 1H); 7.33 | | |
| NMR | (m, 2H); 7.40(m, 1H); 7.53(s, 1H); 7.65(d, J=15.3 Hz, 1H); 8.19(dd, J=8.1, 1.7 Hz, | | |
| | 1H); 8.37(dd, J=4.5, 1.7 Hz, 1H). | | |
| 11.7 | 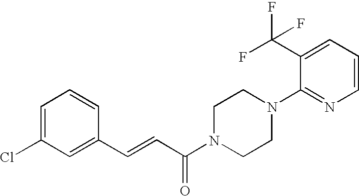 | 1-[(2E)-3-(3-chlorophenyl) prop-2-enoyl]-4-[3-(trifluoro- methyl)pyridin-2-yl]piperazine | 88% |
| | 3.32(m, 4H); 3.80(m, 2H); 3.87(m, 2H); 6.93(d, J=15.3 Hz, 1H); 7.05(m, 1H); 7.29 | | |
| | (m, 2H); 7.37(m, 1H); 7.51(m, 1H); 7.62(d, J=15.3 Hz, 1H); 7.89(dd, J=7.8, 2.4 Hz, | | |
| | 1H); 8.44(m, 1H). | | |
| 11.8 | 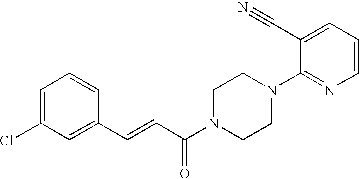 | 2-{4-[(2E)-3-(3-chlorophenyl) prop-2-enoyl]piperazin-1-yl}nicotinonitrile | 83% |
| ¹H | 3.77(m, 4H); 3.85(m, 2H); 3.90(m, 2H); 6.84(dd, J=7.5, 4.8 Hz, 1H); 6.92(d, J=15.3 | | |
| NMR | Hz, 1H); 7.32(m, 2H); 7.36(m, 1H); 7.53(d, J=2.1, 1H), 7.82(dd, J=9.6, 1.9 Hz, 1H); | | |
| | 8.38(dd, J=4.8, 1.9 Hz, 1H). | | |
| 11.9 | 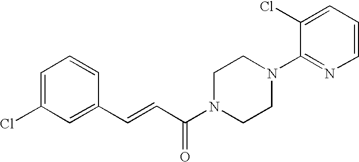 | 1-[(2E)-3-(3-chlorophenyl) prop-2-enoyl]-4-(3-chloro- pyridin-2-yl)piperazine | 75% |
| ¹H | 3.41(m, 4H); 3.83(m, 2H); 3.91(m, 2H); 6.89(dd, J=7.8, 4.8 Hz, 1H); 6.94(d, J=15.3 | | |
| NMR | Hz, 1H); 7.32(m, 2H); 7.36(m, 1H); 7.54(m, 1H), 7.64(d, J=15.3 Hz, 1H); 7.63(dd, J= | | |
| | 7.8, 1.6 Hz, 1H); 8.20(dd, J=4.8, 1.6Hz, 1H). | | |
| 11.10 | 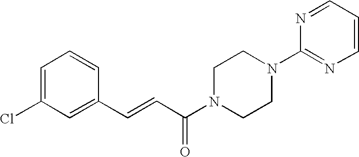 | 2-{4-[(2E)-3-(3-chlorophenyl) prop-2-enoyl]piperazin-1-yl}pyrimidine | 61% |
| ¹H | 8.34(d, 2H), 7.65(d, 1H), 7.53(m, 1H), 7.39(m, 1H), 7.32(m, 2H), 6.93(d, 1H), 6.55(t, | | |
| NMR | 1H), 3.90(br s, 4H), 3.83(br s, 2H), 3.74(br s, 2H). | | |
| 11.11 | 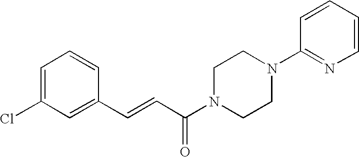 | 1-[(2E)-3-(3-chlorophenyl) prop-2-enoyl]-4-pyridin-2-yl piperazine | 64% |
| ¹H | 3.57(m, 2H); 3.69(m, 2H); 3.78(m, 2H); 3.87(m, 2H); 6.67(m, 2H); 6.93(d, J=15 Hz, | | |
| NMR | 1H); 7.31(m, 2H); 7.48(m, 1H); 7.52(m, 2H); 7.64(d, J=15 Hz); 8.21(m, 1H). | | |
| 11.12 | 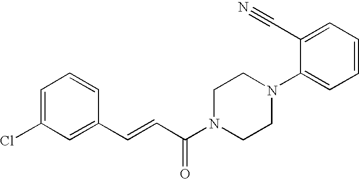 | 2-{4-[(2E)-3-(3-chlorophenyl) prop-2-enoyl]piperazin-1-yl}benzonitrile | quantitative |
| ¹H | 3.23(m, 4H); 3.88(m, 2H); 3.94(m, 4H); 6.93(d, J=15.3 Hz, 1H); 7.01(dm, J=8.1 Hz, | | |
| NMR | 1H); 7.07(td, J=7.5, 0.9 Hz, 1H); 7.30(m, 2H); 7.39(m, 1H); 7.52(m, 2H); 7.59(dd, J= | | |
| | 7.8, 1.6 Hz, 1H); 7.63(d, J=15.3 Hz, 1H). | | |
| 11.13 | 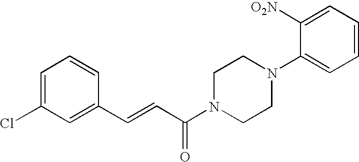 | 1-[(2E)-3-(3-chlorophenyl) prop-2-enoyl]-4-(2-nitro- phenyl)piperazine | quantitative |
| ¹H | 3.12(m, 1H); 3.83(m, 2H); 3.92(m, 2H); 6.92(d,J=15.3 Hz, 1H); 7.15(m, 2H); 7.33 | | |
| NMR | (m, 2H); 7.39(m, 1H); 7.53(m, 2H); 7.64(d, J=15.3 Hz, 1H); 7.82(dd, J=8.1, 1.8 Hz, | | |
| | 1H). | | |
| 11.14 | 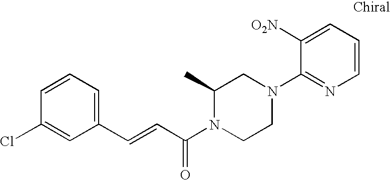 | (2S)-1-[(2E)-3-(3-chloro- phenyl)prop-2-enoyl]-2-methyl- 4-(3-nitropyridin-2-yl) piperazine | 78% |
| | 1.27(m, 3H); 3.12(m, 1H); 3.59(m, 2H); 3.74(m, 2H); 4.22(m, 1H); 6.83(dd, J=7.8, | | |
| | 4.5 Hz, 1H); 6.87(d, J=15.3 Hz, 1H); 7.32(m, 2H); 7.39(m, 1H); 7.53(d, J=2.1 Hz, | | |
| | 1H); 7.65(d, J=15.3 Hz, 1H); 8.17(dd, 3 =8.1, 1.8 Hz, 1H); 8.37(dd, J=4.5, 1.5 Hz, | | |
| | 1H). | | |
| 11.15 | 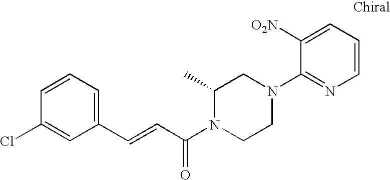 | (2S)-1-[(2E)-3-(3-chloro- phenyl)prop-2-enoyl]-2-methyl- 4-(3-nitropyridin-2-yl) piperazine | 94% |
| | 1.27(m, 3H); 3.12(m, 1H); 3.59(m, 2H); 3.74(m, 2H); 4.22(m, 1H); 6.83(dd, 3 =7.8, | | |
| | 4.5 Hz, 1H); 6.87(d, J=15.3 Hz, 1H); 7.32(m, 2H); 7.39(m, 1H); 7.53(d, J=2.1 Hz, | | |
| | 1H); 7.65(d, 3 =15.3 Hz, 1H); 8.17(dd, 3 =8.1, 1.8 Hz, 1H); 8.37(dd, 3 =4.5, 1.5 Hz, | | |
| | 1H). | | |
| 11.16 | 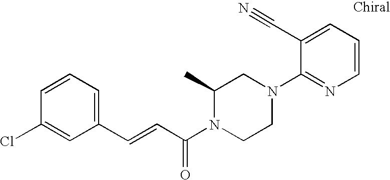 | 2-{(3S)-4-[(2E)-3-(3- chlorophenyl)prop-2-enoyl]-3- methylpiperazin-1-yl}nicotinonitrile | 66% |
| ¹H | 1.41(m, 3H); 3.17(td, J=12.6, 3.3 Hz, 1H); 3.45(m, broad, 2H); 4.21(m, 1H); 4.32(m, | | |
| NMR | 1H); 4.45(m, broad, 1H); 6.82(dd, 3 =7.8, 4.8 Hz, 1H); 6.90(d, 3 =15.3 Hz, 1H); 7.32 | | |
| | (m, 2H); 7.39(m, 1H); 7.53(m, 1H); 7.62(d, 3 =15.3 Hz, 1H); 7.81(dd, 3 =7.8, 1.8 Hz, | | |
| | 1H); 8.37(dd, J=4.8, | | |
| 11.17 | 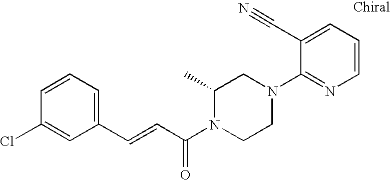 | 2-{(3R)-4-[(2E)-3-(3-chloro- phenyl)prop-2-enoyl]-3- methylpiperazin-1-yl}nicotinonitrile | 71% |
| ¹H | 1.42(m, 3H); 3.18(td, 3 =12.2, 3.3 Hz, 1H); 3.40(m, broad, 2H); 4.22(m, 1H); 4.32(m, | | |
| NMR | 1H); 4.50(m, broad, 1H); 6.83(dd, 3 =7.5, 4.8 Hz, 1H); 6.89(d, 3 =15.3 Hz, 1H); 7.27 | | |
| | (m, 1H); 7.47(m, 2H); 7.64(d, 3 =15.3 Hz, 1H); 7.69(s, 1H); 7.82(dd, 3 =7.5, 1.8 Hz, | | |
| | 1H); 8.37(dd, 3 =4.8, 1.8 Hz, 1H). | | |
| 11.18 | 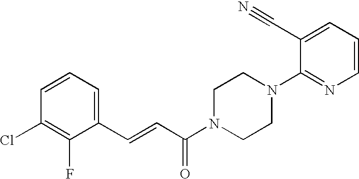 | 2-{4-[(2E)-3-(3-chloro-2- fluorophenyl)prop-2-enoyl]piperazin-1-yl}nicotinonitrile | 84% |
| ¹H | 3.76(m, 4H); 3.81(m, 2H), 3 89(m, 2H); 6.83(dd, J=7.8, 4.8 Hz, 1H); 7.06(d, J=15.6 | | |
| NMR | Hz, 1H); 7.09(m, 1H); 7.37(m, 2H); 7.71(d, J=15.6 Hz, 1H); 7.80(dd, J=7.8, 2.1 Hz | | |
| | 1H); 8.36(dd, J=4.8, 2.1 Hz, 1H). | | |
| 11.19 | 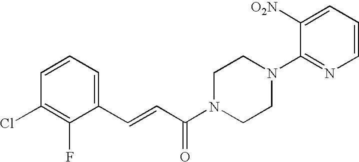 | 1-[(2E)-3-(3-chloro-2-fluoro- phenyl)prop-2-enoyl]-4-(3- nitropyridin-2-yl)piperazine | 62% |
| ¹H | 3.54(m, 4H); 3.82(m, 2H); 3.89(m, 2H); 6.84(dd, J=8.1, 4.5 Hz, 1H); 7.05(d, J=15.6 | | |
| NMR | Hz, 1H); 7.10(m, 1H); 7.38(m, 2H); 7.72(d, J=15.6 Hz, 1H); 8.18(dd, J=8.1, 2.1 Hz, | | |
| | 1H); 8.37(dd, J=4.5, 2.1 Hz, 1H). | | |
| 11.20 | 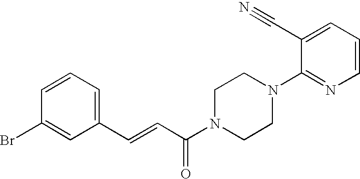 | 2-{4-[(2E)-3-(3-bromo- phenyl)prop-2-enoyl]piperazin-1-yl}nicotinonitrile | 81% |
| ¹H | 3.75(m, 4H); 3.83(m, 2H); 3.87(m, 2H); 6.82(dd, J=7.8, 4.8 Hz, 1H); 6.91(d, J=15.3 | | |
| NMR | Hz, 1H); 7.23(t, J=7.8 Hz, 1H); 7.42(d, J=7.8 Hz, 1H); 7.45(d, J=7.8 Hz, 1H); 7.60 | | |
| | (d, J=15.3 Hz, 1H); 7.67(m, 1H); 7.80(dd, J=7.8, 1.8 Hz, 1H); 8.36(dd, J=4.8, 1.8 | | |
| | Hz, 1H). | | |
| 11.21 | 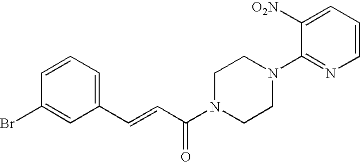 | 1-[(2E)-3-(3-bromo- phenyl)prop-2-enoylI-4-(3- nitropyridin-2-yl)piperazine | 93% |
| ¹H | 3.53(m, 4H); 3.86(m, 4H); 6.83(dd, J=7.8, 4.8 Hz, 1H); 6.89(d, J=15.3 Hz, 1H); 7.24 | | |
| NMR | (t, J=7.8 Hz, 1H); 7.45(m, 2H); 7.62(d, J=15.3 Hz, 1H); 7.67(m, 1H); 8.17(dd, J= | | |
| | 7.8, 1.8 Hz, 1H); 8.37(dd, J=4.8, | | |
| 11.22 | 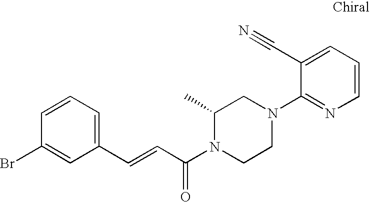 | 2-{(3R)-4-[(2E)-3-(3-bromo- phenyl)prop-2-enoyl]-3- methylpiperazin-1-yl}nicotinonitrile | 71% |
| | 1.41(m, 3H); 3.17(td, J=12.6, 3.3 Hz, 1H); 3.45(m, broad, 2H); 4.21(m, 1H); 4.32(m, | | |
| | 1H); 4.45(m, broad, 1H); 6.82(dd, J=7.8, 4.8 Hz, 1H); 6.90(d, J=15.3 Hz, 1H); 7.32 | | |
| | (m, 2H); 7.39(m, 1H); 7.53(m, 1H); 7.62(d, J=15.3 Hz, 1H); 7.81(dd, J=7.8, 1.8 Hz, | | |
| | 1H); 8.37(dd, J=4.8, 1.8Hz, 1H). | | |
| 11.23 | 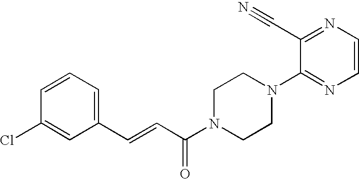 | 3-{4-[(2E)-3-(3-chloro- piperazin-1-yl}pyrazine-2- phenyl)prop-2-enoyl]carbonitrile | 51% |
| ¹H | 3.88(m, 8H); 6.91(d, J=15.3 Hz, 1H); 7.33(m, 2H); 7.40(m, 1H); 7.53(s, 1H); 7.65(d, | | |
| NMR | J=15.3 Hz, 1H); 8.09(d, J=2.1 Hz, 1H); 8.31(d, J=2.1 Hz, 1H). | | |
| 11.24 | 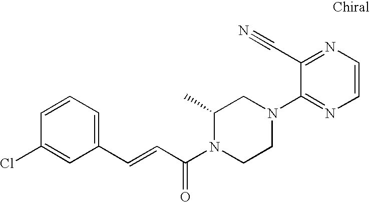 | 3-{(3R)-4-[(2E)-3-(3-chloro- phenyl)prop-2-enoyl]-3- methylpiperazin-1-yl}pyrazine-2-carbonitrile | 67% |
| ¹H | 1.39(m, 3H); 3.28(m, 1H); 3.53(m, 1H); 4.31(m, 1H); 4.41(m, 1H); 6.89(d, J=15.3 | | |
| NMR | Hz, 1H); 7.32(m, 2H); 7.39(m, 1H); 7.52(s, 1H); 7.65(d, J=15.3 Hz, 1H); 8.08(d, J= | | |
| | 2.4 Hz, 1H); 8.29(d, J=2.4 Hz, 1H). | | |
| 11.25 | 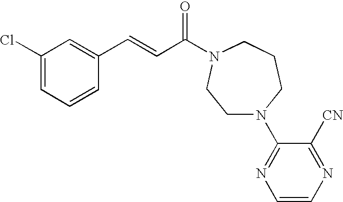 | 3-{4-[(2E)-3-(3-chloro- phenyl)prop-2-enoyl]-1,4- diazepan-1-yl}pyraznie-2- carbonitrile | 92% |
| ¹H | 8.25(m, 1H); 8.01(m, 1H); 7.65(m, 1H); 7.52(m, 1H); 7.33(m, 3H); 6.85(m, 1H); 4.00 | | |
| NMR | (m, 6H); 3.70(m, 2H); 2.15(m, 2H) | | |
| 11.26 | 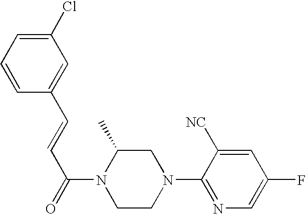 | 2-{(3R)-4-[(2E)-3-(3-chloro- phenyl)prop-2-enoyl]-3- methylpiperazin-1-yl}-5- fluoronicotinonitrile | 23% |
| ¹H | 8.30(m, 1H); 7.60(m, 3H); 7.35(m, 1H); 6.90(d, 1H); 5.20-3.00(m broad, 7H); 1.45(s | | |
| NMR | broad, 3H). | | |
| 11.27 | 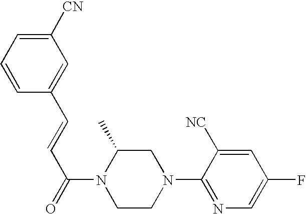 | 2-{(3R)-4-[(2E)-3-(3-cyano- phenyl)prop-2-enoyl]-3- methylpiperazin-1-yl}-5- fluoronicotinonitrile | 19.5% |
| ¹H | 8.30(m, 1H); 7.60(m, 6H); 6.95(d, 1H); 5.2-3.0(m, 7H); 1.41(s broad, 3H) | | |
| NMR | | | |
| 11.28 | 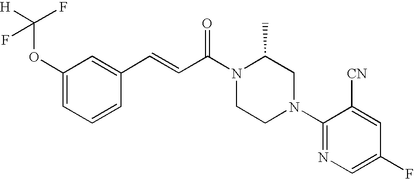 | 2-((3R)-4-{(2E)-3-[3- (difluoromethoxy)phenyl]prop- 2-enoyl}-3-methylpiperazin-1- yl)-5-fluoronicotinonitrile | 20% |
| ¹H | 8.30(m, 1H); 7.60(m, 2H); 7.38(m, 3H); 7.15(m, 1H); 6.80(m, 1H); 5.0-3.0(in broad, | | |
| NMR | 7H); 1.45(s broad, 3H) | | |
| 11.29 | 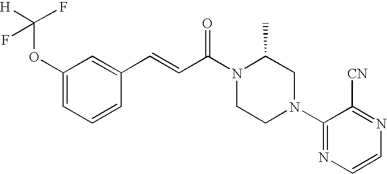 | 2-((3R)-4-{(2E)-3-[3- (difluoromethoxy)phenyl]prop- 2-enoyl}-3-methylpiperazin-1- yl)pyrazine-2-carbonitrile | 31% |
| ¹H | 8.32(m, 1H); 8.10(m, 1H); 7.70(d, 1H); 7.42(m, 2H); 7.28(m, 1H); 7.17(m, 1H); 6.92 | | |
| NMR | (d, 1H); 5.0-3.0(m broad, 7H); 1.30(S broad, 3H) | | |
| 11.30 | 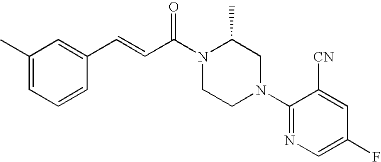 | 5-fluoro-2-{(3R)-3-methyl-4- [(2E)-3-(3-methylphenyl)prop- 2-enoyl]piperazin-1-yl}nicotinonitrile | 22% |
| ¹H | 8.28(m, 1H); 7.41(d, 1H); 7.60(m, 1H); 7.36(m, 2H); 7.27(m, 2H); 6.90(d, 1H); 5-3 | | |
| NMR | (broad m, 7H); 2.40(s, 3H); 1.35(broad s, 3H) | | |
| 11.31 | 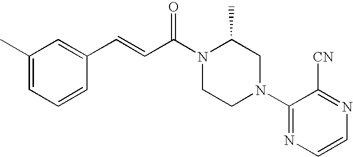 | 3-{(3R)-3-methyl-4-[(2E)-3-(3- methylphenyl)prop-2-enoyl]piperazin-1-yl}pyrazine-2- carbonitrile | 74% |
| ¹H | 8.31(m, 1H); 8.30(m, 1H); 7.75(d, 1H); 7.37(m, 2H); 7.29(m, 2H); 6.88(d, 1H); 5-3 | | |
| NMR | (broad m, 7H); 2.40(s, 3H); 1.40(broad s, 3H) | | |
| 11.32 | 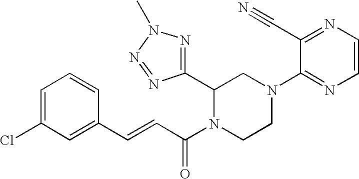 | 4-[(2E)-3-(3-chlorophenyl) prop-2-enoyl]-3-(2-methyl-2H- tetrazol-5-yl)piperazin-1-yl]pyrazine-2-carbonitrile | 65% |
| ¹H | 8.23(d, 1H), 8.04(d, 1H), 7.67(br d, 1H), 7.52(br s, 1H), 7.38(br s, 1H), 7.32(br s, 2H), | | |
| NMR | 6.97(br d, 1H), 6.25(br s, 0.66H), 5.7(br s, 0.34H), 5.03(br d, 1H), 4.6(br m, 0.34H), | | |
| | 4.55(br d, 1H), 4.25(br s, 3H), 4.2(br m, 0.66H) | | |
| 11.33 | 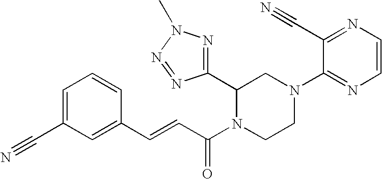 | 3-[4-[(2E)-3-(3-cyanophenyl) prop-2-enoyl]-3-(2-methyl-2H- tetrazol-5-yl)piperazin-1-yl]pyrazine-2-carbonitrile | 58% |
| ¹H | 8.24(d, 1H), 8.05(d, 1H), 7.63-7.85(br d, 4H), 7.51(br s, 1H), 7.06(br d, 1H), 6.25(br s, | | |
| NMR | 0.66H), 5.7(br s, 0.34H), 5(br d, 1H), 4.6(br m, 0.34H), 4.5(br d, 1H), 4.26(br s, 3H), | | |
| | 4.2(br in, 0.66H) | | |
| 11.34 | 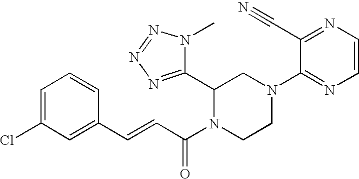 | 3-[4-[(2E)-3-(3-chlorophenyl) prop-2-enoyl]-3-(1-methyl-1H- tetrazol-5-yl)piperazin-1-yl]pyrazine-2-carbonitrile | 63% |
| ¹H | 8.19(d, 1H), 8.03(d, 1H), 7.61(d, 1H), 7.51(s, 1H), 7.38(tin, 1H), 7.32(m, 2H), 6.9(d, | | |
| NMR | 1H), 6.08(t, 1H), 4.79(d, 1H), 4.67(d, 1H), 4.22(s, 3H), 4.1(dd, 1H), 4.02(dd, 1H), 3.67 | | |
| | (m, 2H) | | |
| 11.35 | 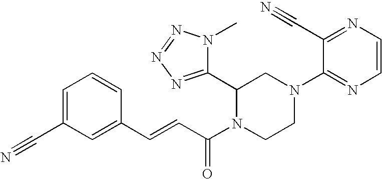 | 3-[4-[(2E)-3-(3-cyanophenyl) prop-2-enoyl]-3-(1-methyl-1H- tetrazol-5-yl)piperazin-1-yl]pyrazine-2-carbonitrile | 67% |

| | |
|---|---|
| ¹H | 8.20(d, 1H), 8.03(d, 1H), 7.85(s, 1H), 7.74(d, 1H), 7.66(s, 1H), 7.63(d, 1H), 7.51(t, |
| NMR | 1H), 7.02(d, 1H), 6.08(t, 1H), 4.80(br d, 1H), 4.68(br d, 1H), 4.24(s +m, 4H), 4.11(dd, |
| | 1H), 4.04(dd, 1H) |

### EXAMPLE 12

### General Procedure: Acryloyl Chloride Coupling to Aryl Piperazine

2-(3-methyl-piperazin-1-yl)-nicotinonitrile (1 mmol) was dissolved in dichloromethane (3 mL) and triethylamine (3 mmol). The solution was cooled to 0° C. and acryloyl chloride (1.5 mmol) was added slowly. The reaction was stiffed for 30 min, then diluted with dichloromethane and quenched with water. The organic phase was dried, filtered and concentrated in vacuo, then chromatographed in ethyl acetate to yield the desired product.

The following compounds were made in this manner:

| Example | Structure | Name | Yield |
|---|---|---|---|
| 12.1 | 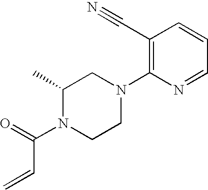 | 2-[(3R)-4-acryloyl-3-methyl- piperazin-1-yl]nicotinonitrile | 64% |
| ¹H | 1.39(d, J=6.6 Hz, 1H); 3.15(td, J=12.3, 3.6 Hz, 1H); 3.38(m, 1H); 4.21(m, 1H); 4.30 | | |
| NMR | (m, 1H); 5.76(dd, J=10, 2 Hz, 1H); 6.36(dd, J=17, 2 Hz, 1H); 6.62(dd, J=17, 10 Hz, | | |
| | 1H); 6.83(dd, J=7.7, 4.8 Hz, 1H); 7.83(dd, J=7.7, 2.0 Hz, 1H); 8.38(dd, J=4.8, 2.0 | | |
| | Hz, 1H). | | |
| 12.2 | 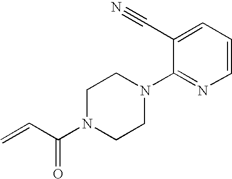 | 2-(4-acryloylpiperazin-1-yl) nicotinonitrile | 67% |
| ¹H | 3.75(m, 6H); 3.88(m, 2H); 5.77(dd, J=10, 2 Hz, 1H); 6.36(dd, J=17, 2 Hz, 1H); 6.62 | | |
| NMR | (dd, J=17, 10 Hz, 1H); 6.85(dd, J=7.5, 4.8 Hz, 1H); 7.83(dd, J=7.5, 2.1 Hz, 1H); 8.39 | | |
| | (dd, J=4.8, 2.1 Hz, 1H). | | |
| 12.3 | 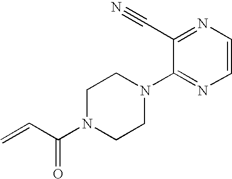 | 3-(4-acryloylpiperazin-1-yl) pyrazine-2-carbonitrile | 63% |

| | |
|---|---|
| ¹H | 8.32(d, 1H); 8.11(d, 1H); 6.61(m, 1H); 6.38(m, 1H); 5.79(m, 1H); 3.84(m, 8H). |
| NMR | |

### EXAMPLE 13

### General Procedure: Cinnamoyl Chloride Coupling to Aryl Piperazine

Oxalyl chloride (2M in dichloromethane, 4 mmol) was added to solution of cinnamic acid (1 mmol) in dichloromethane (10 mL), followed by 1-2 drops of DMF. The reaction mixture stirred at room temperature for 1 hour, then checked for completion by TLC, and concentrated in vacuo to yield the acid chloride.

The cinnamoyl chloride (1 mmol) was added to a cold (0° C.) solution of aryl piperazine (1.2 mmol) in dichloromethane (25 ml) and triethylamine (1 mmol). The reaction mixture was stirred at room temperature for 1 hour, then diluted with dichloromethane and washed with saturated sodium bicarbonate. The organic layer was washed with brine, dried over anhydrous sodium sulfate and concentrated in vacuo. SPE flash column chromatography (silica gel, 0-60% ethyl acetate/hexanes) yielded the desired product.

The following compounds were made in this manner:

| NPS # | Structure | Name | Yield |
|---|---|---|---|
| 13.1 | 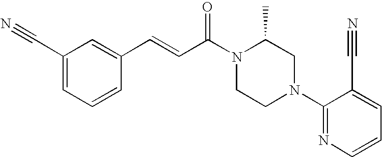 | 2-{(3R)-4-[(2E)-3-(3-cyanophenyl)prop-2- enoyl]-3-methylpiperazin-1-yl}pyridine-3- carbomtrile | 66% |
| ¹H | 8.39(m, 1H); 7.84(m, 2H); 7.69(m, 3H); 7.54(m, 1H); 6.96(m, 1H); 6.85(m, 1H); 4.22 | | |
| NMR | (m, 3H); 3.20(m, 3H); 1.44(d, 3H); 1.28(m, 1H) | | |
| 13.2 | 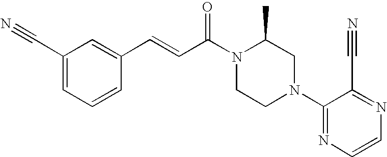 | 3-{(3S)-4-[(2E)-3-(3-cyanophenyl)prop-2- enoyl]-3-methylpiperazin-1-yl}pyrazine-2- carbonitrile | 94% |
| ¹H | 8.32(m, 1H); 8.12(m, 1H); 7.85(m, 1H); 7.71(m, 3H); 7.55(m, 1H); 6.95(m, 1H); 4.40 | | |
| NMR | (m, 3H); 3.41(m, 3H); 1.44(m, 4H) | | |
| 13.3 | 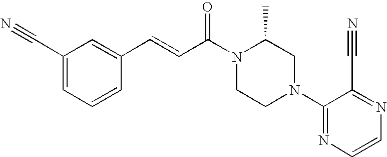 | 3-{(3R)-4-[(2E)-3-(3-cyanophenyl)prop-2- enoyl]-3-methylpiperazin-1-yl}pyrazine-2- carbonitrile | 59% |

| | |
|---|---|
| ¹H | 8.31(m, 1H); 8.11(m, 1H); 7.85(m, 1H); 7.72(m, 3H); 7.54(m, 1H); 6.95(m, 1H); 4.40 |
| NMR | (m, 3H); 3.35(m, 3H); 1.38(m, 4H) |

### EXAMPLE 14

### General Procedure: Palladium Catalyzed Heck Reaction (Conventional Heating)

Sodium acetate (3 mmol), substituted piperazine (1.5 mmol), iodo-benzene or bromo-benzene (1 mmol) and tetrabutylammonium chloride (2 mmol) were dissolved in dimethylacetamide (7 mL) and stirred until all reagents dissolved. Palladium(II) acetate (0.25 mmol) was added and the reaction mixture was stirred at 100° C. for 24 h. The reaction mixture was then cooled to room temperature, diluted with dichloromethane and quenched with water. The organic phase was dried, filtered and concentrated in vacuo, then chromatographed in ethyl acetate in hexanes to yield the desired product.

The following compounds were made in this manner:

| Example | Structure | Name | Yield |
|---|---|---|---|
| 14.1 | 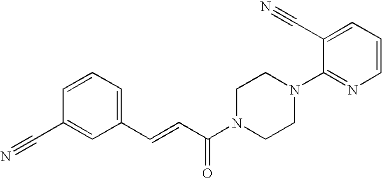 | 2-{4-[(2E)-3-(3-cyanophenyl)prop-2- enoyl]piperazine-1-yl}nicotinonitrile | 53% |
| ¹H | 3.79(m, 4H); 3.86(m, 2H); 3.91(m, 2H); 6.86(dd, J=7.8, 1.8 Hz, 1H); 6.99(d, J=15.3 | | |
| NMR | Hz, 1H); 7.52(t, J=7.8 Hz, 1H); 7.65(d, J=7.8 Hz, 1H); 7.69(d, J=15.3 Hz, 1H); 7.75 | | |
| | (d, J=7.8 Hz, 1H); 7.84(m, 2H); 8.39(dd, J=4.8, 1.8 Hz, 1H). | | |
| 14.2 | 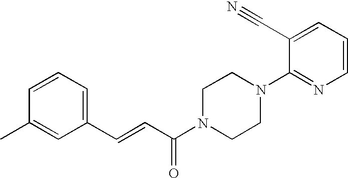 | 2-{4-[(2E)-3-(3-methylphenyl)prop-2- enoyl]piperazin-1-yl}nicotinonitrile | 75% |
| ¹H | 2.39(s, 3H); 3.77(m, 2H); 3.79(m, 2H); 3.90(m, 4H); 6.84(dd, J=7.8, 4.8 Hz, 1H); | | |
| NMR | 6.90(d, J=15.3 Hz, 1H); 7.19(d, J=7.5 Hz, 1H); 7.28(t, J=7.8 Hz, 1H); 7.35(m, 2H); | | |
| | 7.71(d, J=15.3 Hz, 7.83(dd, J=7.5, 2.1 Hz, 1H); 8.39(dd, J=4.8, 2.1 Hz, 1H). | | |
| 14.3 | 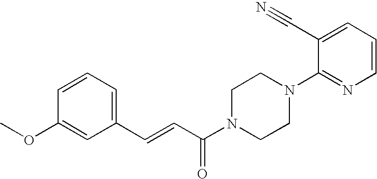 | 2-{4-[(2E)-3-(3-methoxyphenyl)prop- 2-enoyl]piperazin-1-yl}nicotinonitrile | 68% |
| ¹H | 3.76(m, 4H); 3.85(s, 3H), 3 89(m, 4H); 6.84(dd, 3 =7.8, 4.8 Hz, 1H); 6.90(d, J=15.3 | | |
| NMR | Hz, 1H); 6.92(m, 1H); 7.06(m, 1H); 7.14(d, J=7.8 Hz, 1H); 7.31(d, 3 =7.8 Hz, 1H); | | |
| | 7.69(d, J=15.3 Hz, 1H); 7.82(dd, 3 =7.8, 2.0 Hz, 1H); 8.38(4.8, 2.0 Hz, 1H). | | |
| 14.4 | 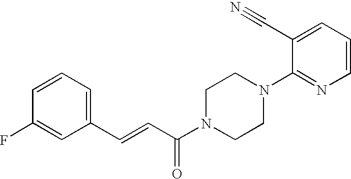 | 2-{4-[(2E)-3-(3-fluorophenyl)prop-2- Fenoyl]piperazin-1 -yl}nicotinonitrile | 71% |
| ¹H | 3.77(m, 4H); 3.84(m, 2H), 3 90(m, 2H); 6.84(dd, 3 =7.5, 4.8 Hz, 1H); 6.97(d, J=15.3 | | |
| NMR | Hz, 1H); 7.06(m, 1H); 7.30(m, 3H); 7.67(d, J=15.3 Hz, 1H); 7.82(dd, J=7.5, 2.1 Hz, | | |
| | 1H); 8.38(dd, 3 =4.8, 2.1 Hz, 1H). | | |
| 14.5 | 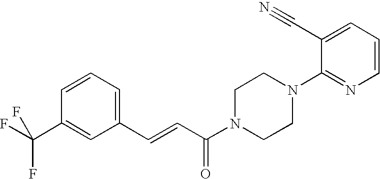 | 2-(4-{(2E)-3-[3-(trifluoromethyl) phenyl]prop-2-enoyl}piperazin-1-yl) nicotinonitrile | 69% |
| ¹H | 3.79(m, 4H); 3.88(m, 2H); 3.91(m, 2H); 6.85(dd, J=7.5, 4.8 Hz, 1H); 6.99(d, 3 =15.3 | | |
| NMR | Hz, 1H); 7.52(t, 3 =7 8 Hz, 1H), 7 62(d, J=7.8 Hz, 1H)); 7.70(m, 1H)); 7.74(d, 3 =15.3 | | |
| | Hz, 1H); 7.81(m, 2H)); 8.39(dd, J=4.8, 2.1 Hz, 1H). | | |
| 14.6 | 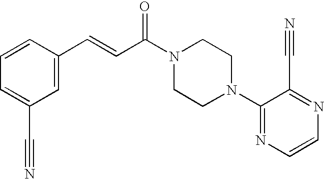 | 3-{4-[(2E)-3-(3-cyanophenyl)prop-2- enoyl]piperazin-1-yl}pyrazine-2- carbomtrile | 16% |
| ¹H | 8.33(d, 1H); 8.13(d, 1H); 7.85(s, 1H); 7.74(d, 1H); 7.69(d, 1H); 7.66(d, 1H); 7.54(m, | | |
| NMR | 1H), 3.90(s, 8H). | | |
| 14.7 | 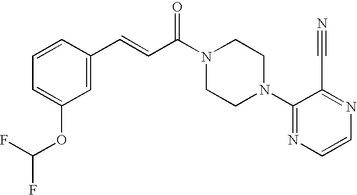 | 3-(4-{(2E)-3-[3-(difluoromethoxy) phenyl]prop-2-enoyl}piperazin-1-yl) pyrazine-2-carbonitrile | 14% |
| ¹H | 8.32(d, 1H); 8.12(d, 1H); 7.71(d, 1H); 7.42(d, 1H); 7.40(s, 1H); 7.29(d, 1H); 6.89(m, | | |
| NMR | 1H); 6.57(d, 1H); 6.32(t, 1H); 3.90(s, 8H). | | |
| 14.8 | 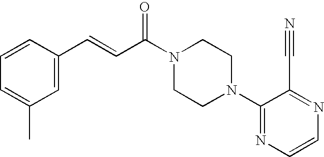 | 3-{4-[(2E)-3-(3-methylphenyl)prop-2- enoyl]piperazin-1-yl}pyrazine-2- carbonitrile | 30% |

| | |
|---|---|
| ¹H | 8.32(d, 1H); 8.11(d, 1H); 7.70(d, 1H); 7.37(d, 1H); 7.35(s, 1H); 7.29(d, 1H), 7.22(m, |
| NMR | 1H); 6.90(d, 1H); 3.90(s, 8H); 2.37(s, 3H). |

### EXAMPLE 15

### General Procedure: Palladium Catalyzed Heck Reaction (Microwave Heating)

Sodium acetate (3 mmol), substituted piperazine (1.5 mmol), iodo-benzene or bromo-benzene (1 mmol), tetrabutylammonium chloride (2 mmol) and palladium(II) acetate (0.25 mmol) were added to a microwave safe vial and dissolved in dimethylacetamide (7 mL). The mixture was stirred at 170° C. in a microwave for 6 min. The reaction mixture was then cooled to room temperature, diluted with dichloromethane and quenched with water. The organic phase was dried, filtered and concentrated in vacuo, then chromatographed in ethyl acetate in hexanes to yield the desired product.

The following compounds were made in this manner:

| Example | Structure | Name | Yield |
|---|---|---|---|
| 15.1 | 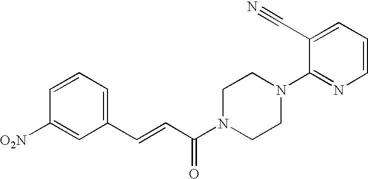 | 2-{4-[(2E)-3-(3-nitrophenyl)prop-2- enoyl]piperazin-1-yl}nicotinonitrile | 26% |
| ¹H | 3.80(m, 4H); 3.89(m, 2H); 3.93(m, 2H); 6.86(m, 1H); 7.07(d, J=15.3 Hz, 1H); 7.60(t, | | |
| NMR | J=7.9 Hz, 1H); 7.76(d, J=15.3 Hz, 1H); 7.84(m, 2H); 8.22(d, J=8.4 Hz, 1H); 8.41 | | |
| | (m, 2H). | | |
| 15.2 | 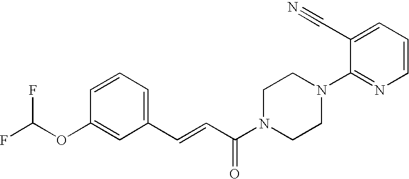 | 2-(4-{(2E)-3-[3-(difluoromethoxy) phenyl]prop-2-enoyl}piperazin-1- yl) meotinonitrile | 41% |
| ¹H | 3.79(m, 4H); 3.86(m, 2H); 3.92(m, 2H); 6.56(t, J=74 Hz, 1H); 6.85(m, 1H); 6.93(d, J= | | |
| NMR | 15.3 Hz, 1H); 7.18(m, 1H); 7.29(d, J=6 Hz, 1H); 7.40(m, 2H); 7.69(d, J=15.3 Hz, | | |
| | 1H); 7.84(m, 1H); 8.39(m, 1H). | | |
| 15.3 | 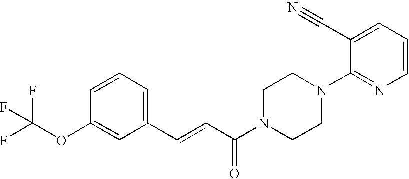 | 2-(4-{(2E)-3-[3-(trifluoromethoxy) phenyl]prop-2-enoyl}piperazin-1- yl) nicotinonitrile | 44% |
| ¹H | 3.79(m, 4H); 3.86(m, 2H); 3.92(m, 2H); 6.85(dd, J=7.5, 4.8 Hz, 1H); 6.94(d, J=15.3 | | |
| NMR | Hz, 1H); 7.24(m, 1H); 7.42(m, 3H); 7.69(d, J=15.3 Hz, 1H); 7.83(dd, J=7.8, 2.1 Hz, | | |
| | 1H); 8.39(dd, J=4.8, 2.1 Hz, 1H). | | |
| 15.4 | 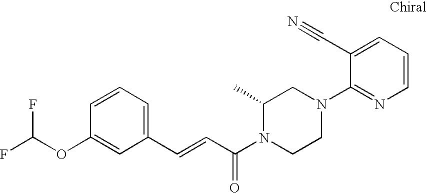 | 2-((3R)-4-{(2E)-3-[3-(difluoro- methoxy)phenyl]prop-2-enoyl}-3- methylpiperazin-1-yl)nicotinonitrile | 32.6% |
| ¹H | 1.42(m, 3H); 3.19(td, J=12.2, 3.6 Hz, 1H); 3.41(m, broad, 2H); 4.23(m, 1H); 4.33(m, | | |
| NMR | 1H); 4.52(m, broad, 1H); 6.56(t, J=73.7 Hz, 1H); 6.82(dd, J=7.5, 4.7 Hz, 1H); 6.90 | | |
| | (d, J=15.6 Hz, 1H); 7.14(m, 1H); 7.30(m, 1H); 7.40(m, 2H); 7.69(d, J=15.6 Hz, 1H); | | |
| | 7.83(dd, J=7.5, 2.1 Hz, 1H); 8.38(dd, J=4.8, 2.1 Hz, 1H). | | |
| 15.5 | 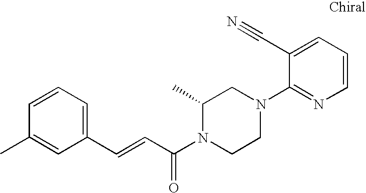 | 2-{(3R)-3-methyl-4-[(2E)-3-(3- methylphenyl)prop-2-enoyl]piperazin-1-yl}nicotinonitrile | 66% |
| ¹H | 1.42(m, 3H); 2.39(s, 3H); 3.11(m, 1H); 3.42(m, broad, 2H); 4.22(m, 1H); 4.37(m, | | |
| NMR | 1H); 6.82(dd, J=7.8, 4.8 Hz, 1H); 6.89(d, J=15.3 Hz, 1H); 7.20(m, 1H); 7.28(m, 1H); | | |
| | 7.34(m, 2H); 7.71(d, J=15.3 Hz, 1H); 7.82(dd, J=7.8, 2.0 Hz, 1H); 8.38(dd, J=4.8, | | |
| | 2.0 Hz, 1H). | | |
| 15.6 | 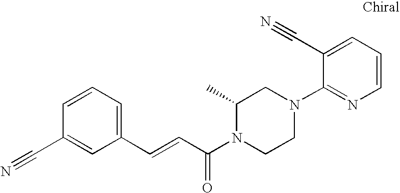 | 2-{(3R)-4-[(2E)-3-(3-cyanophenyl) prop-2-enoyl]-3-methylpiperazin-1- yl}nicotinonitrile | 31% |

| | |
|---|---|
| ¹H | 1.44(m, 3H); 3.19(m, 1H); 3.42(m, broad, 2H); 4.23(m, 1H); 4.34(m, 1H); 6.82(dd, J= |
| NMR | 7.5, 4.8 Hz, 1H); 6.97(d, J=15.3 Hz, 1H); 7.53(t, J=7.8 Hz, 1H); 7.66(m, 1H); 7.70 |
| | (d, 1H); 7.75(m, 1H); 7.83(m, 2H); 8.39(dd, J=4.8, 2.1 Hz, 1H). |

### EXAMPLE 16

### General Procedure: Synthesis of Stabilized Diethyl Phosphonate Intermediate

Piperazine (1 mmol) was dissolved in a solution of (diethoxy-phosphoryl)-acetic acid (1 mmol) in dichloromethane (2 mL). The solution was cooled to −20° C., and TBTU (1 mmol) was added followed by triethylamine (3 mmol). The solution was allowed to warm to room temperature, then stirred overnight. When TLC analysis showed that the reaction was complete, it was diluted with dichloromethane and washed with water. The organic phase was dried, filtered and concentrated, then purified by chromatographing in 10% MeOH in DCM to yield the desired product, contaminated with some tetramethyl urea.

The following compound was made in this manner:

| Example | Structure | Name | Yield |
|---|---|---|---|
| 16.1 | 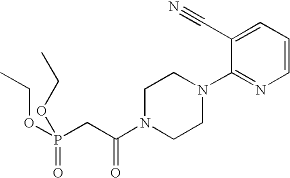 | Diethyl {2-[4-(3-cyanopyridin-2-yl) piperazin-1-yl]-2-oxoethyl}phosphonate | 87% |

| | |
|---|---|
| ¹H | 1.36 (t, J = 7 Hz, 6H); 3.08 (s, 1H); 3.16 (s, 1H); 3.70 (m, 2H); 3.80 (m, 6H); 4.19 (q, J = 7 Hz, |
| NMR | 6.83 (dd, J = 7.8, 4.8 Hz, 1H); 7.82 (dd, J = 7.8, 2.1 Hz, 1H); 8.37 (dd, J = 4.8, 2.1 Hz, 1H). |

### EXAMPLE 17

### General Procedure: Condensation of Aryl Aldehydes with Stabilized Wittig

Sodium hydride (1 mmol, 60% in oil) was stirred in THF (6 mL). Phosphonic acid diethyl ester (1 mmol) was added as a solution in THF (6 mL) and the reaction was stirred for 0.5 h. Aldehyde (1 mmol) was added as a solution in THF (6 mL) and the reaction mixture was heated to 65° C. and stirred overnight. The reaction mixture was then concentrated and chromatographed in ethyl acetate in hexanes to yield the desired product.

The following compounds were made in this manner:

| Example | Structure | Name | Yield |
|---|---|---|---|
| 17.1 | 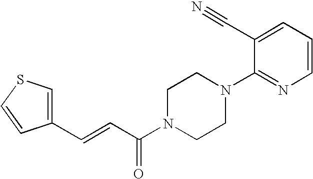 | 2-{4-[(2E)-3-(3-thienyl)prop-2- enoyl]piperazin-1-yl}nicotinonitrile | 24% |
| ¹H | 3.78 (m, 4H); 3.86 (m, 2H); 3.89 (m, 2H); 6.75 (d, J = 15.3 Hz, 1H); 6.85 (dd, J = 7.8, 4.8 Hz, 1H); | | |
| NMR | 7.35 (m, 2H); 7.49 (d, J = 1.2 Hz, 1H); 7.73 (d, J = 15.3 Hz, 1H); 7.83 (dd, J = 7.8, 1.8 Hz, | | |
| | 1H); 8.38 (dd, J = 4.8, 1.8 Hz, 1H). | | |
| 17.2 | 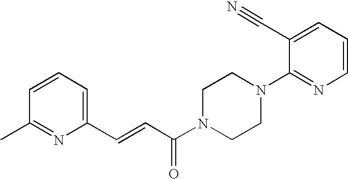 | 2-{4-[(2E)-3-(6-methylpyridin-2- yl)prop-2-enoyl]piperazin-1-yl}nicotinonitrile | 47% |
| ¹H | 3.76 (m, 4H); 3.91 (m, 4H); 6.83 (dd, J = 7.8, 4.8 Hz, 1H); 7.25 (m, 1H); 7.37 (d, J = 7.8 Hz, 1H); | | |
| NMR | 7.56 (d, J = 15 Hz, 1H); 7.69 (d, J = 15 Hz, 1H); 7.72 (d, 1H); 7.82 (dd, J = 7.8, 2.0 Hz, 1H); | | |
| | 8.38 (dd, J = 4.8, 2.0 Hz, 1H); 8.63 (m, 1H). | | |
| 17.3 | 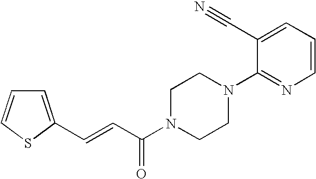 | 2-{4-[(2E)-3-(2-thienyl)prop-2- enoyl]piperazin-1-yl}nicotinonitrile | 46% |

| | |
|---|---|
| ¹H | 3.78 (m, 6H); 3.89 (m, 2H); 6.71 (d, J = 15 Hz, 1H); 6.82 (dd, J = 7.8, 4.8 Hz, 1H); 7.06 (m, 1H); |
| NMR | 7.25 (m, 1H); 7.34 (m, 1H); 7.82 (dd, J = 7.8, 1.8 Hz, 1H); 7.86 (d, J = 15 Hz, 1H); 8.38 (dd, |
| | J = 4.8, 1.8 Hz, 1H). |

### EXAMPLE 18

### 2-(3-chlorophenyl)ethenylboronic acid

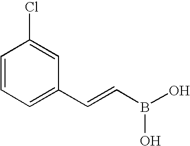

To a vial was added 1-chloro-3-ethynylbenzene (1.0 g, 7.3 mmol) and catecholborane (0.88 g, 7.3 mmol). The mixture was heated at 70° C. for 1.5 hours. Water (4 mL) was added to the reaction mixture and the reaction was heated at 80° C. for 1 hour. The reaction was cooled and filtered and the precipitate was washed with hexanes. The solid was recrystallized from hot water, filtered and dried to yield the boronic acid as an off white solid (0.51 g, 38%).

### EXAMPLE 19

### General Procedure: 3-component coupling of boronic acid, aldehyde & amine

Piperazine (1 mmol), paraformaldehyde (1 mmol) in 1,4-dioxane (3 mL) was heated at 90° C. for ten minutes. Boronic acid (see above) (1.5 mmol) was added followed by 1,4-dioxane (3 mL) and the mixture was heated at 90° C. for thirty minutes. The reaction was cooled and acidified with 2N HCl and the aqueous layer was extracted three times with diethyl ether. It was then basified with 1N NaOH and the product was extracted with diethyl ether. The organic layer was dried over anhydrous sodium sulphate, filtered and concentrated. The product was purified by column chromatography (20%-50% ethyl acetate in hexanes) to yield the desired product.

The following compounds were made in this manner:

| Example | Structure | Name | Yield |
|---|---|---|---|
| 19.1 | 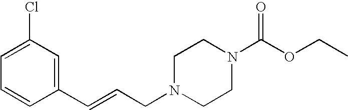 | Ethyl 4-[(2E)-3-(3-chloro- phenyl)prop-2-en-1-yl]piperazine- 1-carboxylate | 26%, colorless oil |
| ¹H | 7.37 (s, 1H), 7.25 (m, 3H), 6.49 (d, 1H), 6.28 (m, 1H), 4.15 (q, 2H), 3.53 (t, 4H), 3.18 (d, 2H), 2.47 (t, 4H), | | |
| NMR | 1.28 (t, 3H) | | |
| 19.2 | 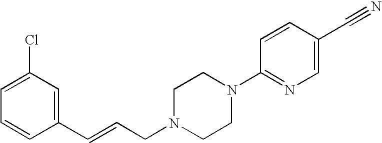 | 6-{4-[(2E)-3-(3- chlorophenyl)prop-2-en-1- yl]piperazin-1-yl} nicotinonitrile | 62%, white solid |

| | |
|---|---|
| ¹H | 8.41 (d, 1H), 7.61 (d of d, 1H), 7.38 (s, 1H), 7.26 (m, 3H), 6.61 (d, 1H), 6.56 (d, 1H), 6.30 (m, 1H), 3.72 (m, 4H), |
| NMR | 3.21 (d of d, 2H), 2.59 (m, 4H). |

### EXAMPLE 20.1

### 4-[3-(3-Chloro-phenyl)-acryloyl]-piperazine-1,3-dicarboxylic acid 1-tert-butyl ester

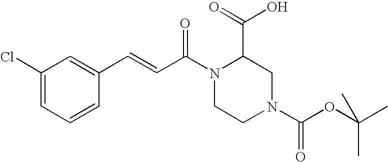

3-Chlorocinnamic acid (2.0 g, 11.29 mmol) was mixed with thionyl chloride (10 mL) and refluxed for 1.5 hours. The reaction mixture was concentrated in vacuo. The residue was dissolved in ThF (10 mL) and added to a mixture of piperazine-1,3-dicarboxylic acid 1-tert-butyl ester (2.0 g, 8.68 mmol) and 2M sodium carbonate (8.7 mL, 17.36 mmol) in THF (30 mL) at 0° C. After 20 min the reaction mixture was concentrated, quenched with water, acidified with 6N HCl. The yellow precipitate was filtered and washed with water. The solid then was dissolved in ethyl acetate and removed water layer, dried with MgSO₄, concentrated. The residue was triturated with 5% ether in hexanes to give 4-[3-(3-chloro-phenyl)-acryloyl]-piperazine-1,3-dicarboxylic acid 1-tert-butyl ester (yellow solid, 3.12 g, 90.9%).

### EXAMPLE 21.1

### 4-[3-(3-Chloro-phenyl)-acryloyl]-piperazine-1,3-dicarboxylic acid 1-tert-butyl ester 3-methyl ester

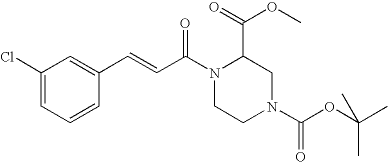

4-[3-(3-Chloro-phenyl)-acryloyl]-piperazine-1,3-dicarboxylic acid 1-tert-butyl ester (3.12 g, 7.9 mmol) was mixed with iodomethane (3.365 g, 23.7 mmol) and potassium carbonate (3.27 g, 23.7 mmol) in DMF (30 mL) at 0° C. for 2 h. The reaction mixture was filtered and washed with dichloromethane. After the filtrate was concentrated to dryness, the residue was diluted with dichloromethane, washed with water and brine, and dried with MgSO4 to give 4-[3-(3-chloro-phenyl)-acryloyl]-piperazine-1,3-dicarboxylic acid 1-tert-butyl ester 3-methyl ester (pale brown sticky oil, 3.15 g, 97.5%).

### EXAMPLE 22.1

### 1-[3-(3-Chloro-phenyl)-acryloyl]-piperazine-2-carboxylic acid methyl ester

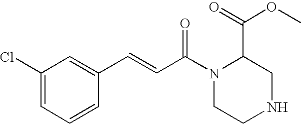

Trifluoroacetic acid (10 mL) was added carefully to a solution 4-[3-(3-chloro-phenyl)-acryloyl]-piperazine-1,3-dicarboxylic acid 1-tert-butyl ester 3-methyl ester (3.15 g, 7.7 mmol) in dichloromethane (10 mL) at 0° C. The reaction mixture was stirred for 1 h and concentrated. The residue was dissolved in water and extracted with ethyl acetate to remove impurities. The aqueous layer was basified with 2M sodium carbonate and extracted with ethyl acetate again. The organic layer was dried, concentrated to give 1-[3-(3-chloro-phenyl)-acryloyl]-piperazine-2-carboxylic acid methyl ester (pale yellow sticky oil, 1.6 g, 67.2%).

### EXAMPLE 23.1

### 4-[3-(3-Chloro-phenyl)-acryloyl]-3′-cyano-3,4,5,6-tetrahydro-2H-[1,2′]bipyrazinyl-3-carboxylic acid methyl ester

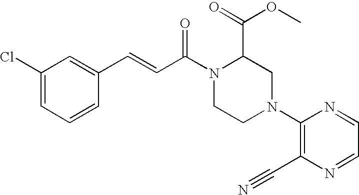

1-[3-(3-Chloro-phenyl)-acryloyl]-piperazine-2-carboxylic acid methyl ester (1.6 g, 5.18 mmol) was mixed with 3-chloro-pyrazine-2-carbonitrile (0.868 g, 6.22 mmol) and triethylamine (1.05 g, 10.36 mmol) in acetonitrile at 90° C. overnight. The reaction mixture was quenched water and extracted with dichloromethane. The product was purified by column chromatography with 50% ethyl acetate in hexanes to give 4-[3-(3-chloro-phenyl)-acryloyl]-3′-cyano-3,4,5,6-tetrahydro-2H-[1,2′]bipyrazinyl-3-carboxylic acid methyl ester (white solid, 1.5 g, 70.3%).

### EXAMPLE 24.1

### 4-[3-(3-Chloro-phenyl)-acryloyl]-3′-cyano-3,4,5,6-tetrahydro-2H-[1,2]bipyrazinyl-3-carboxylic acid

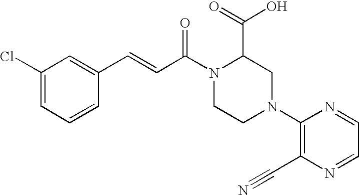

4-[3-(3-Chloro-phenyl)-acryloyl]-3′-cyano-3,4,5,6-tetrahydro-2H-[1,2′]bipyrazinyl-3-carboxylic acid methyl ester (411.9 mg, 1.0 mmol) was mixed with 0.5 N LiOH (4.8 mL, 2.4 mmol) in THF (2.4 mL) at 45° C. for 20 minutes. The reaction mixture was diluted with water and acidified with 3N HCl to pH3, extracted with ethyl acetate, dried, concentrated. The residue was triturated with 10% ether in hexanes to give 4-[3-(3-chloro-phenyl)-acryloyl]-3′-cyano-3,4,5,6-tetrahydro-2H-[1,2′]bipyrazinyl-3-carboxylic acid (white solid, 369.9 mg, 92.9%).

### EXAMPLE 25.1

### 4-[3-(3-Chloro-phenyl)-acryloyl]-3′-cyano-3,4,5,6-tetrahydro-2H-[1,2]bipyrazinyl-3-carboxylic acid amide

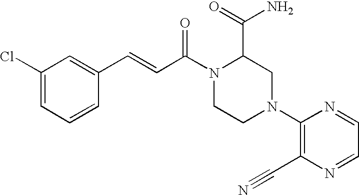

Isobutyl chloroformate (104 mg, 0.761 mmol) was added dropwise to a solution of 4-[3-(3-chloro-phenyl)-acryloyl]-3′-cyano-3,4,5,6-tetrahydro-2H-[1,2′]bipyrazinyl-3-carboxylic acid (198.9 mg, 0.5 mmol) and triethylamine (209 μL, 1.5 mmol) in THF (6 mL) at −70° C., and reaction mixture was stirred for 15 minutes. Concentrated ammonia solution (2 mL) was added and the reaction temperature was raised to 0° C. The reaction mixture was diluted with water and extracted with ethyl acetate. The product was purified by column chromatography with 20˜80% ethyl acetate in hexanes to give 4-[3-(3-chloro-phenyl)-acryloyl]-3′-cyano-3,4,5,6-tetrahydro-2H-[1,2′]bipyrazinyl-3-carboxylic acid amide (white solid, 80 mg, 40.3%).

### EXAMPLE 26.1

### 4-[3-(3-Chloro-phenyl)-acryloyl]-3,4,5,6-tetrahydro-2H-[1,2]bipyrazinyl-3,3′-dicarbonitrile

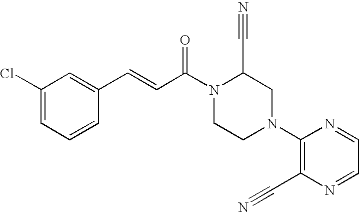

4-[3-(3-chloro-phenyl)-acryloyl]-3′-cyano-3,4,5,6-tetrahydro-2H-[1,2′]bipyrazinyl-3-carboxylic acid amide (35 mg, 0.088 mmol) was mixed with cyanuric acid (9.74 mg, 0.528 mmol) in DMS (100 μL) at room temperature for 30 min. The reaction mixture was quenched with water and extracted with ethyl acetate. The organic layer was washed with saturated sodium bicarbonate and dried with sodium sulfate. The product was triturated with 5% ethyl acetate in ether to give 4-[3-(3-chloro-phenyl)-acryloyl]-3,4,5,6-tetrahydro-2H-[1,2′]bipyrazinyl-3,3′-dicarbonitrile (white solid, 27 mg, 81%).

### EXAMPLE 27.1

### 4-[3-(3-Chloro-phenyl)-acryloyl]-3-hydroxymethyl-3,4,5,6-tetrahydro-2H-[1,2]bipyrazinyl-3′-carbonitrile

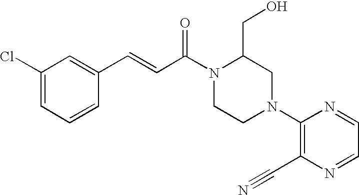

Isobutyl chloroformate (55.4 mg, 0.406 mmol) was added dropwise to a solution of 4-[3-(3-chloro-phenyl)-acryloyl]-3′-cyano-3,4,5,6-tetrahydro-2H-[1,2]bipyrazinyl-3-carboxylic acid (146.7 mg, 0.3687 mmol) and triethylamine (153.9 μL, 1.1 mmol) in THF (6 mL) at −70° C., and reaction mixture was stirred for 30 minutes. Sodium borohydride (27.9 mg, 0.737 mmol) in water (200 μL) was added. The reaction temperature was raised to 0° C. and the mixture was quenched with 1N HCl to pH3. Ethyl acetate was used to extract product. The organic layer was washed with brine and the product was purified by column chromatography with 50% ethyl acetate in hexanes to give 4-[3-(3-Chloro-phenyl)-acryloyl]-3-hydroxymethyl-3,4,5,6-tetrahydro-2H-[1,2′]bipyrazinyl-3′-carbonitrile (white solid, 44 mg, 31%).

### EXAMPLE 28.1

### 3-[4-[(2E)-3-(3-chlorophenyl)prop-2-enoyl]-3-(fluoromethyl)piperazin-1-yl]pyrazine-2-carbonitrile

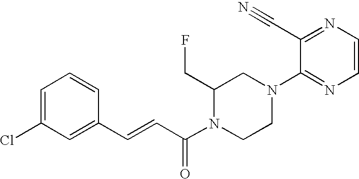

A solution of 3-[4-[(2E)-3-(3-chlorophenyl)prop-2-enoyl]-3-(hydroxymethyl)piperazin-1-yl]pyrazine-2-carbonitrile (32 mg, 0.083 mmol) and DAST (20 mg, 0.125 mmol) in dichloromethane (3 mL) was stirred at −70° C., then warmed to room temperature for 1 h. The reaction quenched with water and the product was extracted into ethyl acetate. The organic layer was washed with sodium bicarbonate (3×), dried and evaporated in vacuo. Chromatography (50-80% ethyl acetate in hexanes) gave the title compound (yellow sticky oil, 5.5 mg, 17%).

### EXAMPLE 29.1

### 3-[4-[(2E)-3-(3-chlorophenyl)prop-2-enoyl]-3-(methoxymethyl)piperazin-1-yl]pyrazine-2-carbonitrile

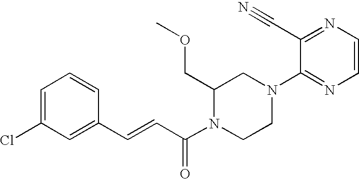

A solution of 3-[4-[(2E)-3-(3-chlorophenyl)prop-2-enoyl]-3-(hydroxymethyl)piperazin-1-yl]pyrazine-2-carbonitrile (180 mg, 0.47 mmol) and sodium tert-butoxide (496 mg, 0.52 mmol) in THF (3 mL) was heated at 40-45° C.

After 5 minutes iodomethane (114 mg, 0.94 mmol) was added. The reaction mixture was stirred at 40-45° C. overnight prior to quenching with water and extraction with ethyl acetate. The organic layer was washed with water, dried (silica gel), filtered and evaporated in vacuo. Chromatography gave the title compound (pale yellow solid, 120 mg, 64%).

### EXAMPLE 30

### Pharmaceutical Examples

Functional Assessment of mGluR5 Antagonism in Cell Lines Expressing mGluR5D

The properties of the compounds of the invention can be analyzed using standard assays for pharmacological activity. Examples of glutamate receptor assays are well known in the art as described in for example Aramori et al., Neuron 8:757 (1992), Tanabe et al., Neuron 8:169 (1992), Miller et al., J. Neuroscience 15: 6103 (1995), Balazs, et al., J. Neurochemistry 69:151 (1997). The methodology described in these publications is incorporated herein by reference. Conveniently, the compounds of the invention can be studied by means of an assay (FLIPR) that measures the mobilization of intracellular calcium, [Ca²⁺]ᵢ in cells expressing mGluR5 or another assay (IP3) that measures inositol phosphate turnover.

FLIPR Assay

Cells expressing human mGluR5d as described in WO97/05252 are seeded at a density of 100,000 cells per well on collagen coated clear bottom 96-well plates with black sides and experiments are done 24 h following seeding. All assays are done in a buffer containing 127 mM NaCl, 5 mM KCl, 2 mM MgCl₂, 0.7 mM NaH₂PO₄, 2 mM CaCl₂, 0.422 mg/ml NaHCO₃, 2.4 mg/ml HEPES, 1.8 mg/ml glucose and 1 mg/ml BSA Fraction IV (pH 7.4). Cell cultures in the 96-well plates are loaded for 60 minutes in the above mentioned buffer containing 4 μM of the acetoxymethyl ester form of the fluorescent calcium indicator fluo-3 (Molecular Probes, Eugene, Oreg.) in 0.01% pluronic acid (a proprietary, non-ionic surfactant polyol—CAS Number 9003-11-6). Following the loading period the fluo-3 buffer is removed and replaced with fresh assay buffer. FLIPR experiments are done using a laser setting of 0.800 W and a 0.4 second CCD camera shutter speed with excitation and emission wavelengths of 488 nm and 562 nm, respectively. Each experiment is initiated with 160 μl of buffer present in each well of the cell plate. A 40 μl addition from the antagonist plate was followed by a 50 μL addition from the agonist plate. A 90 second interval separates the antagonist and agonist additions. The fluorescence signal is sampled 50 times at 1 second intervals followed by 3 samples at 5 second intervals immediately after each of the two additions. Responses are measured as the difference between the peak height of the response to agonist, less the background fluorescence within the sample period. IC₅₀ determinations are made using a linear least squares fitting program.

IP3 Assay

An additional functional assay for mGluR5d is described in WO97/05252 and is based on phosphatidylinositol turnover. Receptor activation stimulates phospholipase C activity and leads to increased formation of inositol 1,4,5,triphosphate (IP₃).

GHEK stably expressing the human mGluR5d are seeded onto 24 well poly-L-lysine coated plates at 40×10⁴ cells/well in media containing 1 μCi/well [3H]myo-inositol. Cells were incubated overnight (16 h), then washed three times and incubated for 1 h at 37° C. in HEPES buffered saline (146 mM NaCl, 4.2 mM KCl, 0.5 mM MgCl₂, 0.1% glucose, 20 mM HEPES, pH 7.4) supplemented with 1 unit/ml glutamate pyruvate transaminase and 2 mM pyruvate. Cells are washed once in HEPES buffered saline and pre-incubated for 10 min in HEPES buffered saline containing 10 mM LiCl. Compounds are incubated in duplicate at 37° C. for 15 min, then either glutamate (80 μM) or DHPG (30 μM) is added and incubated for an additional 30 min. The reaction is terminated by the addition of 0.5 ml perchloric acid (5%) on ice, with incubation at 4° C. for at least 30 min. Samples are collected in 15 ml polyproplylene tubes and inositol phosphates are separated using ion-exchange resin (Dowex AG1-X8 formate form, 200-400 mesh, BIORAD) columns. Inositol phosphate separation was done by first eluting glycero phosphatidyl inositol with 8 ml 30 mM ammonium formate. Next, total inositol phosphates is eluted with 8 ml 700 mM ammonium formate/100 mM formic acid and collected in scintillation vials. This eluate is then mixed with 8 ml of scintillant and [3H] inositol incorporation is determined by scintillation counting. The dpm counts from the duplicate samples are plotted and IC₅₀ determinations are generated using a linear least squares fitting program.

Generally, the compounds of the present invention were active in the assays described herein at concentrations (or with IC₅₀ values) of less than 10 μM. Preferred compounds of the invention have IC₅₀ values of less than 1 μM; more preferred compounds of less than about 100 nM. For example, the compounds of Examples 11.6, 11.22, 11.28, 29.1 and 11.4 have IC₅₀ values of 10, 33, 293, 384 and 1113 nM, respectively.

What is claimed is:

1. A compound of formula I or a pharmaceutically acceptable salt or solvate thereof: 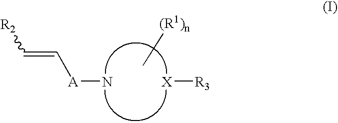
wherein 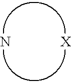
is a 5- to 7-membered ring that optionally contains, in addition to N and X, 1 to 2 heteroatoms that are independently selected from the group consisting of N, S, and O, wherein 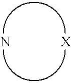
is optionally fused to a 5- to 7-membered ring that optionally contains one or more heteroatoms independently selected from the group consisting of N, O and S;
R¹, in each instance, is independently selected from the group consisting of F, Cl, Br, I, OH, CN, nitro, C₁₋₆-alkyl, OC₁₋₆-alkyl, C₁₋₆-alkylhalo, OC₁₋₆-alkylhalo, C₂₋₆-alkenyl, OC₂₋₆-alkenyl, C₂₋₆-alkynyl, OC₂₋₆-alkynyl, C₃₋₈-cycloalkyl, OC₃₋₈-cycloalkyl, C₁₋₆-alkylene-C₃₋₈-cycloalkyl, OC₁₋₆-alkylene-C₃₋₈-cycloalkyl, C₃₋₈-heterocycloalkyl, OC₃₋₈-heterocycloalkyl, C₁₋₆-alkylene-C₃₋₈-heterocycloalkyl, OC₁₋₆-alkylene-C₃₋₈-heterocycloalkyl, aryl, heteroaryl, C₁₋₆-alkylenearyl, C₁₋₆-alkyleneheteroaryl, OC₁₋₆-alkylenearyl, OC₁₋₆-alkyleneheteroaryl, C₁₋₆-alkyleneheterocycloalkyl, OC₁₋₆-alkyleneheterocycloalkyl, (CO)R⁴, O(CO)R⁴, O(CO)OR⁴, CO₂R⁴, O(CNR⁵)OR⁴, C₁₋₆-alkyleneOR⁴, OC₂₋₆-alkyleneOR⁴, C₁₋₆-alkylene(CO)R⁴, OC₁₋₆-alkylene(CO)R⁴, C₁₋₆-alkyleneCO₂R⁴, OC₁₋₆-alkyleneCO₂R⁴, C₁₋₆-alkylenecyano, OC₂₋₆-alkylenecyano, C₀₋₆-alkyleneNR⁴R⁵, OC₂₋₆-alkyleneNR⁴R⁵, C₁₋₆-alkylene(CO)NR⁴R⁵, OC₁₋₆-alkylene(CO)NR⁴R⁵, C₀₋₆-alkyleneNR⁴(CO)R⁵, OC₂₋₆-alkyleneNR⁴(CO)R⁵, C₀₋₆-alkyleneNR⁴(CO)NR⁴R⁵, OC₁₋₆-alkyleneNR⁴(CO)NR⁴R⁵, C₀₋₆-alkyleneSR⁴, OC₂₋₆-alkyleneSR⁴, C₀₋₆-alkylene(SO)R⁴, OC₂₋₆-alkylene(SO)R⁴, C₀₋₄-alkyleneSO₂R⁴, OC₂₋₆-alkyleneSO₂R⁴, C₀₋₆-alkylene(SO₂)NR⁴R⁵, OC₂₋₆-alkylene(SO₂)NR⁴R⁵, C₀₋₆-alkyleneNR⁴(SO₂)R⁵, OC₂₋₆-alkyleneNR⁴(SO₂)R⁵, C₀₋₆-alkyleneNR⁴(SO₂)NR⁴R⁵, OC₂₋₆-alkyleneNR⁴(SO₂)NR⁴R⁵, (CO)NR⁴R⁵, O(CO)NR⁴R⁵, NR⁴OR⁵, C₁₋₆-alkyleneNR⁴OR⁵, OC₁₋₆-alkyleneNR⁴OR⁵, C₀₋₆-alkyleneNR⁴(CO)OR⁵, OC₂₋₆-alkyleneNR⁴(CO)OR⁵ and SO₃R⁴;
R² is a 5- to 7-membered ring that optionally contains one or more heteroatoms that are independently selected from the group consisting of N, O and S, wherein the 5- to 7-membered ring is optionally substituted with one or more substituents that are selected from the group consisting of F, Cl, Br, I, OH, nitro, C₁₋₆-alkyl, C₁₋₆-alkylhalo, OC₁₋₆-alkyl, OC₁₋₆-alkylhalo, —CN, aryl, heteroaryl, CO₂R⁴, NR⁴R⁵, SR⁴, S(O)R⁴ and SO₂R⁴;
R³ is selected from the group consisting of:
C(Y)YR⁴;
phenyl that is substituted at the 2-position, 4-position, or both, with one or more substituents selected from the group consisting of F, Cl, Br, I, nitro, C₁₋₆-alkyl, C₁₋₆-alkylhalo, OC₁₋₆-alkyl, OC₁₋₆-alkylhalo, and —CN; and
a 5- or 6-membered heterocyclic ring having a N atom in the ring at the position adjacent to the point of attachment and said ring is optionally substituted with one or more substituents selected from the group consisting of F, Cl, Br, I, OH, nitro, C₁₋₆-alkyl, C₁₋₆-alkylhalo, OC₁₋₆-alkyl, OC₁₋₆-alkylhalo, —CN, aryl, heteroaryl, CO₂R⁴, SR⁴, S(O)R⁴ and SO₂R⁴, wherein when R³ is a 6-membered ring the substituents are located para to the point of attachment, or ortho to the point of attachment or both.
R⁴ and R⁵ are independently selected from the group consisting of H, C₁₋₆-alkyl, C₁₋₆-alkylhalo, C₂₋₆-alkenyl, C₂₋₆-alkynyl, C₃₋₈-cycloalkyl, C₁₋₆-alkyl-C₃₋₈-cycloalkyl, aryl, C₁₋₆-alkylenearyl, C₁₋₆-alkylene-heterocycloalkyl, heteroaryl and C₁₋₆alkyleneheteroaryl, wherein any cyclic moiety is optionally fused to a 5- to 7-membered ring that may contain one or more heteroatoms independently selected from the group consisting of C, N, O and S;
A is selected from the group consisting of CR⁴R⁵—, —C(O)—, —C(NR⁴)— and —C(S)—;
X is selected from the group consisting of C and N, wherein when X is carbon, then X, together with one adjacent carbon atom in the ring 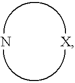
may form a double bond;
Y is selected from the group consisting of O, NR⁴ and S; and
n is an integer that is selected from the group consisting of 0, 1, 2, 3, 4, and 5;
with the proviso that the compound is not:
1-[4-(3-Nitro-pyridin-2-yl)-piperazin-1-yl]-3-phenyl-propenone.
1-[4-(3-methyl-2-pyridinyl)-piperazin-1-yl]-3-(4-trifluoromethylphenyl)-prop-2-en-1-one, and
1-[4-(3-trifluoromethyl-2-pyridinyl)-piperazin-1-yl]-3-(4-isopropylphenyl)-prop-2-en-1-one.

2. The compound according to claim 1, wherein X is N.

3. The compound according to claim 1, wherein 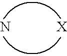
is piperazinyl.

4. The compound according to claim 1, wherein R² is selected from the group consisting of optionally substituted phenyl, pyridyl, and thienyl.

5. The compound according to claim 1, wherein R² is phenyl that is substituted with one or more substituents that are selected from the group consisting of F, Cl, Br, nitro, C₁₋₆-alkyl, C₁₋₆-alkylhalo, OC₁₋₆-alkyl, OC₁₋₆-alkylhalo, and —CN.

6. The compound according to claim 1, wherein R² is optionally substituted pyridyl.

7. The compound according to claim 6, wherein R² is optionally substituted 2-pyridyl.

8. The compound according to claim 1, wherein R² is optionally substituted thienyl.

9. The compound according to claim 8, wherein R₂ is optionally substituted 3-thienyl.

10. The compound according to claim 1, wherein R¹ is selected from the group consisting of C₁₋₆-alkyl, C₁₋₆-haloalkyl, —CN, —CO₂R₄, —CONR₄R₅, —C₁₋₆alkyleneOR₄, —(CH₂)nNR₄, —C₁₋₆-alkyleneCO₂R₄, and the following rings:

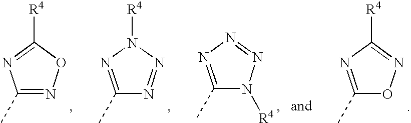

11. The compound according to claim 1, wherein R⁴ and R⁵ are independently selected from H and C₁₋₆-alkyl.

12. The compound according to claim 1, wherein R¹ is selected from the group consisting of C₁₋₆-alkyl, CN and CO₂R⁴, where R⁴ is C₁₋₆-alkyl.

13. The compound according to claim 1, wherein n is 0 or 1.

14. The compound according to claim 1, wherein R³ is phenyl that is substituted at the 2-position, 4-position, or both, with one or more substituents selected from the group consisting of F, Cl, Br, I, nitro, C₁₋₆-alkyl, C₁₋₆-alkylhalo, OC₁₋₆-alkyl, OC₁₋₆-alkylhalo, and —CN.

15. The compound according to claim 14, wherein R³ is phenyl that is substituted at the 2-position.

16. The compound according to claim 1, wherein R³ is a 5- or 6-membered heterocyclic ring having an N atom in the ring adjacent to the point of attachment wherein said ring is optionally substituted with one or more substituents selected from the group consisting of F, Cl, Br, I, OH, nitro, C₁₋₆alkyl, c₁₋₆alkylhalo, OC₁₋₆-alkyl, OC₁₋₆-alkylhalo, —CN, aryl, heteroaryl, CO₂R⁴, SR⁴, S(O)R⁴ and SO₂R⁴ and when R³ is a 6-membered ring the substituents are located para to the point of attachment or ortho to the point of attachment or both.

17. The compound according to claim 16, wherein the substituents are selected from the group consisting of F, Cl, Br, I, nitro, C₁₋₆-alkyl, C₁₋₆-alkylhalo, OC₁₋₆-alkyl, OC₁₋₆-alkylhalo, and —CN.

18. The compound according to claim 17, wherein R³ is selected from the group consisting of optionally substituted 2-pyrazinyl.

19. The compound according to claim 1, wherein R³ is optionally substituted 2-pyridyl.

20. The compound according to claim 1, wherein: 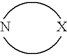
is piperazinyl;
R¹ is selected from the group consisting of C₁₋₆-alkyl, C₁₋₆-haloalkyl, heteroaryl, —CN, —CO₂R₄, —CONR₄R₅, —C₁₋₆-alkyl-OR₄, —(CH₂)ₙNR₄, —C₁₋₆-alkyl-CO₂R₄;
R² is selected from the group consisting of optionally substituted phenyl, pyridyl, and thienyl;
R³ is a 2-pyridyl or 2-pyrazinyl ring that is optionally substituted in the position para to the point of attachment or ortho to the point of attachment or both, with one or more substituents selected from the group consisting of F, Cl, Br, I, nitro, C₁₋₆-alkyl, C₁₋₆-alkylhalo, OC₁₋₆-alkyl, OC₁₋₆-alkylhalo, and —CN;
A is —C(O)—; and
the double bond to which R² is bound is in the E configuration.

21. A compound selected from the group consisting of:
Ethyl 4-[(2E)-3-(4-chloro-phenyl prop-2-enoyl]piperazine-1-carboxylate,
6-{4-[(2E)-3-(4-chlorophenyl)prop-2-enoyl]piperazin-1-yl}nicotinonitrile,
1-[(2E)-3-(4-chlorophenyl)prop-2-enoyl]-4-(3-nitro-pyridin-2-yl)piperazine,
ethyl 4-[(2E)-3-(3-chloro-phenyl)prop-2-enoyl]piperazine-1-carboxylate,
6-{4-[(2E)-3-(3-chloro-phenyl)prop-2-enoyl]piperazin-1-yl}nicotinonitrile,
1-[(2E)-3-(3-chlorophenyl)prop-2-enoyl]-4-(3-nitro-pyridin-2-yl)piperazine,
1-[(2E)-3-(3-chlorophenyl)prop-2-enoyl]-4-[3-(trifluoro-methyl)pyridin-2-yl]piperazine,
2-{4-[(2E)-3-(3-chlorophenyl)prop-2-enoyl]piperazin-1-yl}nicotinonitrile,
1-[(2E)-3-(3-chlorophenyl)prop-2-enoyl]-4-(3-chloro-pyridin-2-yl)piperazine,
2-{4-[(2E)-3-(3-chlorophenyl)prop-2-enoyl]piperazin-1-yl}pyrimidine,
1-[(2E)-3-(3-chlorophenyl)prop-2-enoyl]-4-pyridin-2-yl piperazine,
2-{4-[(2E)-3-(3-chlorophenyl)prop-2-enoyl]piperazin-1-yl}benzonitrile,
1-[(2E)-3-(3-chlorophenyl)prop-2-enoyl]-4-(2-nitro-phenyl)piperazine,
(2S)-1-[(2E)-3-(3-chloro-phenyl)prop-2-enoyl]-2-methyl-4-(3-nitropyridin-2-yl)piperazine,
(2R)-1-[(2E)-3-(3-chloro-phenyl)prop-2-enoyl]-2-methyl-4-(3-nitropyridin-2-yl)piperazine,
2-{(3S)-4-[(2E)-3-(3-chlorophenyl)prop-2-enoyl]-3-methylpiperazin-1-yl}nicotinonitrile,
2-{(3R)-4-[(2E)-3-(3-chloro-phenyl)prop-2-enoyl]-3-methylpiperazin-1-yl}nicotinonitrile,
2-{4-[(2E)-3-(3-chloro-2-fluorophenyl)prop-2-enoyl]piperazin-1-yl}nicotinonitrile,
1-[(2E)-3-(3-chloro-2-fluoro-phenyl)prop-2-enoyl]-4-(3-nitropyridin-2-yl)piperazine,
2-{4-[(2E)-3-(3-bromo-phenyl)prop-2-enoyl]piperazin-1-yl}nicotinonitrile,
1-[(2E)-3-(3-bromo-phenyl)prop-2-enoyl]-4-(3-nitropyridin-2-yl)piperazine,
2-{(3R)-4-[(2E)-3-(3-bromo-phenyl)prop-2-enoyl]-3-methylpiperazin-1-yl}nicotinonitrile,
3-{4-[(2E)-3-(3-chloro-phenyl)prop-2-enoyl]piperazin-1-yl}pyrazine-2-carbonitrile,
3-{(3R)-4-[(2E)-3-(3-chloro-phenyl)prop-2-enoyl]-3-methylpiperazin-1-yl}pyrazine-2-carbonitrile,
3-{4-[(2E)-3-(3-chloro-phenyl)prop-2-enoyl]-1,4-diazepan-1-yl}pyrazine-2-carbonitrile,
2-{(3R)-4-[(2E)-3-(3-chloro-phenyl)prop-2-enoyl]-3-methylpiperazin-1-yl}-5-fluoronicotinonitrile,
2-{(3R)-4-[(2E)-3-(3-cyano-phenyl)prop-2-enoyl]-3-methylpiperazin-1-yl}-5-fluoronicotinonitrile,
2-((3R)-4-{(2E)-3-[3-(difluoromethoxy)phenyl]prop-2-enoyl}-3-methylpiperazin-1-yl)-5-fluoronicotinonitrile,
3-((3R)-4-{(2E)-3-[3-(difluoromethoxy)phenyl]prop-2-enoyl}-3-methylpiperazin-1-yl)pyrazine-2-carbonitrile,
5-fluoro-2-{(3R)-3-methyl-4-[(2E)-3-(3-methylphenyl)prop-2-enoyl]piperazin-1-yl}nicotinonitrile,
3-{(3R)-3-methyl-4-[(2E)-3-(3-methylphenyl)prop-2-enoyl]piperazin-1-yl}pyrazine-2-carbonitrile,
3-[4-[(2E)-3-(3-chlorophenyl)prop-2-enoyl]-3-(2-methyl-2H-tetrazol-5-yl)piperazin-1-yl]pyrazine-2-carbonitrile,
3-[4-[(2E)-3-(3-cyanophenyl)prop-2-enoyl]-3-(2-methyl-2H-tetrazol-5-yl)piperazin-1-yl]pyrazine-2-carbonitrile,
3-[4-[(2E)-3-(3-chlorophenyl)prop-2-enoyl]-3-(1-methyl-1H-tetrazol-5-yl)piperazin-1-yl]pyrazine-2-carbonitrile,
3-[4-[(2E)-3-(3-cyanophenyl)prop-2-enoyl]-3-(1-methyl-1H-tetrazol-5-yl)piperazin-1-yl]pyrazine-2-carbonitrile,
2-{(3R)-4-[(2E)-3-(3-cyanophenyl)prop-2-enoyl]-3-methylpiperazin-1-yl}pyridine-3-carbonitrile,
3-{(3S)-4-[(2E)-3-(3-cyanophenyl)prop-2-enoyl]-3-methylpiperazin-1-yl}pyrazine-2-carbonitrile,
3-{(3R)-4-[(2E)-3-(3-cyanophenyl)prop-2-enoyl]-3-methylpiperazin-1-yl}pyrazine-2-carbonitrile,
2-{4-[(2E)-3-(3-cyanophenyl)prop-2-enoyl]piperazine-1-yl}nicotinonitrile,
2-{4-[(2E)-3-(3-methylphenyl)prop-2-enoyl]piperazin-1-yl}nicotinonitrile,
2-{4-[(2E)-3-(3-methoxyphenyl)prop-2-enoyl]piperazin-1-yl}nicotinonitrile,
2-{4-[(2E)-3-(3-fluorophenyl)prop-2-enoyl]piperazin-1-yl}nicotinonitrile,
2-(4-{(2E)-3-[3-(trifluoromethyl)phenyl]prop-2-enoyl}piperazin-1-yl)nicotinonitrile,
3-{4-[(2E)-3-(3-cyanophenyl)prop-2-enoyl]piperazin-1-yl}pyrazine-2-carbonitrile,
3-(4-{(2E)-3-[3-(difluoromethoxy)phenyl]prop-2-enoyl}piperazin-1-yl)pyrazine-2-carbonitrile,
3-{4-[(2E)-3-(3-methylphenyl)prop-2-enoyl]piperazin-1-yl}pyrazine-2-carbonitrile,
2-{4-[(2E)-3-(3-nitrophenyl)prop-2-enoyl]piperazin-1-yl}nicotinonitrile,
2-(4-{(2E)-3-[3-(difluoromethoxy)phenyl]prop-2-enoyl}piperazin-1-yl)nicotinonitrile,
2-(4-{(2E)-3-[3-(trifluoromethoxy)phenyl]prop-2-enoyl}piperazin-1-yl)nicotinonitrile,
2-((3R)-4-{(2E)-3-[3-(difluoro-methoxy)phenyl]prop-2-enoyl}-3-methylpiperazin-1-yl)nicotinonitrile,
2-{(3R)-3-methyl-4-[(2E)-3-(3-methylphenyl)prop-2-enoyl]piperazin-1-yl}nicotinonitrile,
2-{(3R)-4-[(2E)-3-(3-cyanophenyl)prop-2-enoyl]-3-methylpiperazin-1-yl}nicotinonitrile,
Diethyl {2-[4-(3-cyanopyridin-2-yl)piperazin-1-yl]-2-oxoethyl}phosphonate,
2-{4-[(2E)-3-(3-thienyl)prop-2-enoyl]piperazin-1-yl}nicotinonitrile,
2-{4-[(2E)-3-(6-methylpyridin-2-yl)prop-2-enoyl]piperazin-1-yl}nicotinonitrile,
2-{4-[(2E)-3-(2-thienyl)prop-2-enoyl]piperazin-1-yl}nicotinonitrile,
Ethyl 4-[(2E)-3-(3-chloro-phenyl)prop-2-en-1-yl]piperazine-1-carboxylate,
6-{4-[(2E)-3-(3-chlorophenyl)prop-2-en-1-yl]piperazin-1-yl}nicotinonitrile,
4-[3-(3-Chloro-phenyl)-acryloyl]-3′-cyano-3,4,5,6-tetrahydro-2H-[1,2′]bipyrazinyl-3-carboxylic acid methyl ester,
4-[3-(3-Chloro-phenyl)-acryloyl]-3′-cyano-3,4,5,6-tetrahydro-2H-[1,2′]bipyrazinyl-3-carboxylic acid,
4-[3-(3-Chloro-phenyl)-acryloyl]-3,4,5,6-tetrhydro-2H-[1,2′]bipyrazinyl-3,3′-dicarbonitrile,
4-[3-(3-Chloro-phenyl)-acryloyl]-3-hydroxymethyl-3,4,5,6-tetrahydro-2H-[1,2′]bipyrazinyl-3′-carbonitrile,
3-[4-[(2E)-3-(3-chlorophenyl)prop-2-enoyl]-3-(fluoromethyl)piperazin-1-yl]pyrazine-2-carbonitrile, and
3-[4-[(2E)-3-(3-chlorophenyl)prop-2-enoyl]-3-(methoxymethyl)piperazin-1-yl]pyrazine-2-carbonitrile.

22. A pharmaceutical composition comprising as active ingredient a therapeutically effective amount of the compound according to claim 1, in association with one or more pharmaceutically acceptable diluents, excipients and/or inert carriers.

23. The compound according to claim 1 for use in therapy.

24. The compound according to claim 1 for use in treatment of mGluR 5 mediated disorders.

25. Use of the compound according to claim 1, in the manufacture of a medicament for the treatment of mGluR5 mediated disorders.

26. A method of treatment of mGluR5 mediated disorders, comprising administering to a mammal a therapeutically effective amount of the compound according to claim 1.

27. The method according to claim 26, wherein the mammal is a human.

28. The method according to claim 27, wherein the disorders are neurological disorders.

29. The method according to claim 27, wherein the disorders are psychiatric disorders.

30. The method according to claim 27, wherein the disorders are chronic and acute pain disorders.

31. The method according to claim 27, wherein the disorders are gastrointestinal disorders.
